(19) United States
(12) Patent Application Publication
(10) Pub. No.: US 2026/0226550 A1
(43) Pub. Date: Aug. 6, 2026

(54) METHODS AND SYSTEMS FOR TREATMENT DETERMINATION AND ANALYSIS

(71) Applicant: AMPEL BioSolutions, LLC, Charlottesville, VA (US)

(72) Inventors: Peter Lipsky, Charlottesville, VA (US); Prathyusha Bachali, Seattle, WA (US); Andrea Daamen, Charlottesville, VA (US); Amrie Grammer, Charlottesville, VA (US)

(21) Appl. No.: 19/531,346

(22) Filed: Feb. 5, 2026

Related U.S. Application Data
(60) Provisional application No. 63/754,773, filed on Feb. 6, 2025.

(51) Int. Cl. C12Q 1/6883; A61K 31/5377; C12Q 1/6869

(52) U.S. Cl. CPC C12Q 1/6883; A61K 31/5377; C12Q 1/6869; C12Q 2600/106; C12Q 2600/158

(57) ABSTRACT

The present invention provides methods for monitoring treatment of a subject. The subject may be suffering from an autoimmune condition (e.g. lupus). The methods may comprise analyzing expression levels of genes of a plurality of gene sets to determine an endotype. A treatment dose may be determined based on the endotype.

### CROSS-REFERENCE

This application claims the benefit of U.S. Provisional Application No. 63/754,773, filed Feb. 6, 2025, which application is incorporated herein by reference in its entirety.

### BACKGROUND

Many diseases may have heterogeneous patient populations. These patient populations may have differences in phenotype, endotype, or a combination thereof. Heterogenous patient populations may benefit from personalized treatment approaches.

### SUMMARY

In an aspect, the present disclosure provides a method of monitoring treatment of a subject, the method comprising: (a) providing a first sample from the subject having received a first dose of a cereblon E3 ligase modulator; (b) analyzing expression levels of genes of a plurality of gene sets in the first sample to determine a first endotype, wherein the plurality of gene sets comprises at least one gene set comprising at least 30% of genes of a gene set from Table 1; and (c) based at least in part on the first endotype, determining a second dose of a cereblon E3 ligase modulator for the subject.

In some embodiments, the method further comprises, before (a) providing a second sample from the subject. In some embodiments, the method further comprises analyzing expression levels of genes of the plurality of gene sets in the second sample to determine a second endotype. In some embodiments, the method further comprises, based at least in part on the second endotype, determining the first dose of the cereblon E3 ligase modulator for the subject. In some embodiments, the method further comprises administering the first dose of the cereblon E3 ligase modulator to the subject. In some embodiments, the method further comprises obtaining the first sample from the subject. In some embodiments, the method further comprises, after (a), processing the first sample to generate an enriched sample. In some embodiments, the processing comprises enriching the first sample for at least a subset of the genes in the plurality of gene sets. In some embodiments, the enriching comprises using a probe set to bind to the at least subset of the genes in the plurality of gene sets. In some embodiments, the method further comprises, after (c), administering the second dose of the cereblon E3 ligase modulator to the subject. In some embodiments, the method further comprises, after (c), delivering a report to a care provider comprising information regarding the second dose of the cereblon E3 ligase modulator. In some embodiments, (b) comprises using a cloud computing system to determine the first endotype. In some embodiments, the subject is suffering from an autoimmune disease. In some embodiments, the autoimmune disease is lupus. In some embodiments, the lupus is systemic lupus erythematosus (SLE). In some embodiments, (b) comprises performing RNA-seq. In some embodiments, performing RNA-seq comprises sequencing sequences to generate at least 50,000 sequence reads. In some embodiments, the method further comprises aligning the at least 50,000 sequence reads against a reference sequence. In some embodiments, the plurality of gene sets comprises at least 6 gene sets. In some embodiments, the first sample comprises a blood sample, a tissue sample, or a biopsy sample. In some embodiments, the first sample is a processed blood sample. In some embodiments, the processed blood sample comprises cell lysate from white blood cells, cell-free RNA, or a combination thereof. In some embodiments, the first dose of the cereblon E3 ligase modulator is the same as the second dose of cereblon E3 ligase modulator. In some embodiments, the first dose of the cereblon E3 ligase modulator is higher than the second dose of cereblon E3 ligase modulator. In some embodiments, the first dose of the cereblon E3 ligase modulator is lower than the second dose of cereblon E3 ligase modulator. In some embodiments, the cereblon E3 ligase modulator comprises thalidomide, lenalidomide, pomalidomide, mezigdomide, iberdomide, golcadomide, or an analogue thereof. In some embodiments, the cereblon E3 ligase modulator comprises Iberdomide. In some embodiments, the cereblon E3 ligase modulator is an oral dose of the cereblon E3 ligase modulator.

In an aspect, the present disclosure provides a method of determining a treatment for a subject, the method comprising: (a) providing a sample from the subject, wherein the subject is suffering from lupus; (b) analyzing expression levels of genes of a plurality of gene sets in the sample to determine an endotype, wherein the plurality of gene sets comprises at least one gene set comprising at least 30% of genes of a gene set from Table 1; and (c) based at least on part on the endotype of the subject, determining an initial dose of a cereblon E3 ligase modulator for the subject.

In some embodiments, the method further comprises, after (c), administering the initial dose of the cereblon E3 ligase modulator to the subject. In some embodiments, the method further comprises, after (c), delivering a report to a care provider comprising information regarding the second dose of the cereblon E3 ligase modulator. In some embodiments, the lupus is systemic lupus erythematosus (SLE). In some embodiments, (b) comprises performing RNA-seq. In some embodiments, performing RNA-seq comprises sequencing sequences to generate at least 50,000 sequence reads. In some embodiments, the method further comprises aligning the at least 50,000 sequence reads against a reference sequence. In some embodiments, the method further comprises obtaining the sample from the subject. In some embodiments, the method further comprises after (a), processing the sample to generate an enriched sample. In some embodiments, the processing comprises enriching the sample for at least a subset of the genes in the plurality of gene sets. In some embodiments, the enriching comprises using a probe set to specifically bind to the at least subset of the plurality of gene sets. In some embodiments, the sample comprises a blood sample, a tissue sample, or a biopsy sample. In some embodiments, the sample is a processed blood sample. In some embodiments, the processed blood sample comprises cell lysate from white blood cells, cell-free RNA, or a combination thereof. In some embodiments, the initial dose of cereblon E3 ligase modulator comprises thalidomide, lenalidomide, pomalidomide, mezigdomide, iberdomide, golcadomide, or an analogue thereof. method of claim 29, wherein the initial dose of cereblon E3 ligase modulator comprises Iberdomide. In some embodiments, the initial dose of the cereblon E3 ligase modulator is an oral dose of the cereblon E3 ligase modulator.

As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a heatmap of gene GSVA scores for endotype subsets.

FIG. 2 shows a bar graph showing the molecular endotype subset for various treatment arms.

FIG. 3 shows heatmaps of gene expression profiles in SLE subjects.

FIG. 4 shows a heat map, table, and graphs showing gene expression profiles and treatment information.

FIG. 5 shows heatmaps of contributing features in a random forest model prediction.

FIG. 6 shows graphs and charts of changes in gene expression.

FIG. 7 shows plots of k-means clustering results and principal component analysis.

FIG. 8 shows plots of machine learning analysis of gene set variation analysis.

FIG. 9 shows a heatmap of gene GSVA scores for endotype subsets.

FIG. 10 shows a diagram of a computer system.

FIG. 11 shows a matrix of a comparison of a random forest classifier model and a K-means clustering approach.

FIG. 12 shows a bar graph showing the molecular endotype subset for various treatment arms.

FIG. 13 shows heatmaps of gene expression changes.

FIG. 14 shows heatmaps of contributing features in a random forest model prediction.

FIG. 15 shows a diagram of an endotype determination pipeline.

### DETAILED DESCRIPTION

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Methods

The present disclosure provides methods for determining a treatment, monitoring a treatment, adjusting a treatment, administering a treatment, processing a sample, or a combination thereof. The methods described herein may be useful for determining treatment regimen or dosage for a subject based on their biology (e.g. their endotype). For example, the methods described herein may be useful for determining an initial dosage of a drug for treatment of a condition in a subject based on the gene expression of genes in multiple gene sets from a sample of the subject. In some cases, the methods may involve determining an initial treatment (e.g. dosage) of a subject. In some cases, the methods may involve determining a treatment dose for a subject that has previously received an initial dose.

An aspect of the present disclosure provides a method of monitoring treatment of a subject. The method may comprise providing a sample (e.g. a first sample) from the subject. The subject may have received a dose (e.g. a first dose) of a treatment (e.g. a cereblon E3 ligase modulator). The method may comprise analyzing expression levels of genes of a plurality of gene sets in the sample (e.g. the first sample) to determine an endotype (e.g. a first endotype). The plurality of gene sets may comprise at least one gene set comprising at least 30% of genes of a gene set from Table 1. The method may comprise determining a second dose of a treatment (e.g. a cereblon E3 ligase modulator) for the subject based at least in part on the first endotype.

Another aspect of the present disclosure provides a method of determining a treatment for a subject. The method may comprise providing a sample from the subject. The subject may be suffering from a condition (e.g. lupus). The method may comprise analyzing expression levels of genes of a plurality of gene sets in the sample to determine an endotype. The plurality of gene sets may comprise at least one gene set comprising at least 30% of genes of a gene set from Table 1. The method may comprise determining an initial dose (e.g. a first dose) of a treatment (e.g. a cereblon E3 ligase modulator) for the subject based at least on part on the endotype of the subject.

In some cases, the methods of the present disclosure may comprise providing one or more additional samples (e.g. a second sample) from the subject. The one or more additional samples may comprise one or more of the samples as described herein. For example, a first sample may comprise a blood sample from the subject and a second sample may comprise a blood sample from the subject. In some cases, one or more additional samples may be the same sample type as the sample from the subject (e.g. the first sample) or a different sample type as the sample from the subject (e.g. the first sample. The methods may comprise obtaining one or more samples. For example, the methods described herein may comprise extracting one or more samples from the subject. As another example, the methods may comprise receiving one or more samples from the subject (e.g. receiving from a medical care provider or laboratory testing facility one or more samples from the subject).

The methods described herein may comprise processing one or more samples as described herein. Processing the one or more samples may comprise a variety of sample treatments and/or manipulations. For example, processing the sample may comprise enriching the sample for one or more components (e.g. for one or more nucleic acids). In some cases, the enriching may comprise selectively binding, sorting, filtering, capturing, and/or concentrating a subset of nucleic acids in the sample. The subset of nucleic acids in the sample may comprise genes of interest (e.g. genes present in one or more gene modules that may be used for determining a subject's endotype.) In some cases, enriching the sample for a subset of nucleic acids may comprise using a probe set (e.g. an oligonucleotide set) to bind to at least a subset of genes). Processing the one or more samples may comprise filtering the one or more samples, centrifuging the one or more samples, lysing the one or more samples, heating the one or more samples, cooling the one or more samples, vortexing the one or more samples, mixing the sample, or any combination thereof.

The methods described herein may comprise analyzing expression levels of genes from a sample from the subject (e.g. a first sample). In some cases, the expression levels of genes of a plurality gene sets may be analyzed. For example, the methods may comprise analyzing the expression levels of genes of a plurality of gene sets in a first sample (e.g. the first sample), in a second sample (e.g. the second sample), in one or more additional samples, or any combination thereof. Analyzing gene expression levels of genes (e.g. genes of a plurality of gene sets) may be used to determine an endotype of the subject. For example, a sample from a subject may be analyzed to measure gene expression of genes from a plurality of gene sets. Based on the gene expression levels, an endotype of the subject may be determined. The endotype may be associated with a medical condition. The endotype may be used to determine a treatment recommendation (e.g. a dosage and/or a treatment type). For example, an endotype of the subject may be used to determine a first dose of a cereblon E3 ligase modulator, a second dose of a cereblon E3 ligase modulator, an initial dose (e.g. first does) of an E3 ligase modulator, or any combination thereof. In some cases, the cereblon E3 ligase modulator may comprise iberdomide.

In some cases, the methods described herein may comprise administering one or more doses of one or more treatments to the subject. In some cases, the methods described herein may comprise administering a first dose, a second dose, an initial does, or a combination thereof to the subject. The one or more treatments may comprise any one of the treatments described herein. In some cases, the one or more treatment may comprise a drug. The drug may comprise a cereblon E3 ligase modulator (e.g. iberdomide). The one or more doses may comprise a quantity and/or concentration of one or more treatments (e.g. drugs). For example, a dose comprising 1 milligram (mg) of a drug per kilogram (kg) of a subject may be administered to the subject. Administering dose of a treatment to a subject may be performed in a number of ways, including but not limited to orally, sublingually, topically, through inhalation, intravenously, intramuscularly, subcutaneously, or any combination thereof. In some cases, the cereblon E3 ligase modulator may be an oral dose of the cereblon E3 ligase modulator.

In some cases, a subject may have received one or more doses. For example, a subject may have received a first dose (e.g. an initial dose), a second dose, a third dose, a third dose, a fourth dose, or more doses. In some cases, a subject may have received a first dose of a cereblon E3 ligase modulator (e.g. a first dose of iberdomide) and a second dose of a cereblon E3 ligase modulator (e.g. a second dose of iberdomide). The first dose of the cereblon E3 ligase modulator may be the same (e.g. the same quantity) as the second dose of the cereblon E3 ligase modulator. The first dose of the cereblon E3 ligase modulator may be higher than the second dose of the cereblon E3 ligase modulator. The first dose of the cereblon E3 ligase modulator may be lower than the second dose of the cereblon E3 ligase modulator.

The methods described herein may comprise generating a report. Generating a report may be performed before a subject is treated, after a subject is treated, or a combination thereof. The report may comprise a variety of information, including but not limited to personal identification information (e.g. name, date of birth, identification number), age, medical information, treatment information, genetic information, lab results, analysis of gene expression information (e.g. endotype information), dosing information, counseling information (e.g. medical recommendations). The report may provide (e.g. delivered) to any number of parties, including but not limited to a subject, a family member, a care giver, a medical provider, a doctor's office, a hospital, or a combination thereof. The report may be delivered digitally, by mail, or a combination thereof. In some cases, the methods described herein may comprise delivering a report to a care provider. The report may comprise information regarding a dose (e.g. a first dose and/or a second dose) of a cereblon E3 ligase modulator (e.g. iberdomide).

### Conditions

The methods described herein may involve processing and/or analyzing samples from a subject, determining treatment for a subject, administering treatment for a subject, adjusting a treatment for a subject, determining an endotype or a subject, or any combination thereof. The subject may be a human subject. The subject of the methods described herein may be suffering from a condition. The condition may comprise an autoimmune condition, an inflammatory condition, a chronic condition, or a combination thereof. The autoimmune condition may comprise alopecia areata, amyloidosis, ankylosing spondylitis, anti-NMDA receptor encephalitis, antiphospholipid syndrome, aplastic anemia, arteriosclerosis, autoimmune Addison's disease, autoimmune angioedema, autoimmune autonomic ganglionopathy, autoimmune encephalitis/acute disseminated encephalomyelitis (ADEM), autoimmune gastritis, autoimmune hemolytic anemia, autoimmune hepatitis, autoimmune hyperlipidemia, autoimmune hypophysitis/lymphocytic hypophysitis, autoimmune inner ear disease, autoimmune interstitial lung disease, autoimmune lymphoproliferative syndrome, autoimmune myelofibrosis, autoimmune myocarditis, autoimmune oophoritis, autoimmune pancreatitis, autoimmune polyglandular syndromes, autoimmune progesterone dermatitis, autoimmune retinopathy, autoimmune sudden sensorineural hearing loss, balo disease/concentric sclerosis, behçet's disease, birdshot chorioretinopathy, bullous pemphigoid, castleman disease, celiac disease, chagas disease, chronic autoimmune urticaria, chronic inflammatory demyelinating polyneuropathy, churg-strauss syndrome, cogan's syndrome, cold agglutinin disease, crest syndrome, crohn's disease, cronkhite-canada syndrome, cryptogenic organizing pneumonia, dermatitis herpetiformis, dermatomyositis, discoid lupus, dressler's syndrome, eczema/atopic dermatitis, endometriosis, eosinophilic esophagitis/eosinophilic gastroenteritis, eosinophilic fasciitis, erythema nodosum, essential mixed cryoglobulinemia, evans syndrome, fibromyalgia, fibrosing alveolitis/idiopathic pulmonary fibrosis, giant cell arteritis/temporal arteritis/horton's disease, giant cell myocarditis, glomerulonephritis, goodpasture's syndrome/anti-gbm/anti-tbm disease, granulomatosis with polyangiitis/wegener's granulomatosis, graves' disease, guillain-barrè syndrome, hashimoto's thyroiditis/autoimmune thyroiditis, henoch-schonlein purpura, hidradenitis suppurativa, hurst's disease/acute hemorrhagic leukoencephalitis, hypogammaglobulinemia, iga nephropathy, igg4-related sclerosing disease, immune thrombocytopenia/autoimmune thrombocytopenia purpura, immune-mediated necrotizing myopathy, inclusion body myositis, interstitial cystitis, juvenile idiopathic arthritis/adult-onset still's disease, juvenile myositis, kawasaki disease, lambert-eaton myasthenic syndrome, leukocytoclastic vasculitis, lichen planus, lichen sclerosus, linear iga disease, lupus nephritis, lyme disease, ménière's disease, microscopic polyangiitis/ANCA-associated vasculitis, mixed connective tissue disease, mucous membrane pemphigoid/ocular cicatricial pemphigoid, multiple sclerosis, myalgic encephalomyelitis/chronic fatigue syndrome, myasthenia gravis, myelin oligodendrocyte glycoprotein antibody disease (MOGAD), narcolepsy, neuromyelitis optica/device's disease, optic neuritis, palindromic rheumatism, palmoplantar pustulosis, paraneoplastic cerebellar degeneration, paraneoplastic pemphigus, paroxysmal nocturnal hemoglobinuria, parry-romberg syndrome/hemifacial atrophy/progressive facial hemiatrophy, parsonage-turner syndrome, pemphigoid gestationis/herpes gestationis, pemphigus foliaceus, pemphigus vulgaris, pernicious anemia, pityriasis lichenoides et varioliformis acuta (PLEVA)/mucha-habermann disease, poems syndrome, polyarteritis nodosa, polymyalgia rheumatica, polymyositis, postural orthostatic tachycardia syndrome, primary biliary cholangitis (PBC), primary sclerosing cholangitis, psoriasis, psoriatic arthritis, pure red cell aplasia, pyoderma gangrenosum, raynaud's syndrome, reactive arthritis, relapsing polychondritis, rheumatic fever, rheumatoid arthritis, sarcoidosis, schmidt syndrome, scleritis, scleroderma/systemic sclerosis, sjögren disease (SjD), small fiber sensory neuropathy, sydenham's chorea, systemic lupus erythematosus, takayasu arteritis, testicular autoimmunity/autoimmune orchitis, thyroid eye disease/graves' ophthalmopathy/thyroid-associated orbitopathy, transverse myelitis, type 1 diabetes/insulin-dependent diabetes, ulcerative colitis, undifferentiated connective tissue disease, vitiligo, or a combination thereof. The autoimmune condition may be lupus. The lupus may be systemic lupus erythematosus (SLE), cutaneous (skin) lupus, drug-induced lupus, neonatal lupus, or a combination thereof. In some cases, the subject is suffering from SLE.

The subject of the methods described herein may be suffering from one or more symptoms associated with one or more conditions. In some cases the subject may be suffering from fatigue, malaise, joint pain, muscle pain, skin abnormalities, low-grade fever, digestion issues, neurological issues, cognitive issues, dry eyes, dry mouth, dry skin, hair loss, swollen glands, weight gain, weight loss, or any combination thereof. Conditions relevant to the methods described herein are described in International Application Nos. PCT/US2023/020752, and PCT/US2024/010125, each of which is incorporated herein by reference in its entirety for any purpose.

### Treatments

The methods described herein may involve one or more treatments. In some cases, the methods may comprise monitoring a treatment in a subject, determining a treatment for a subject, adjusting a treatment for a subject based one or more endotypes of the subject, administering a treatment (e.g. a dose of a treatment) to a subject, or any combination thereof. The one or more treatments of the methods described herein may comprise one or more doses of the same treatment, one or more doses of a different treatment, or a combination thereof. In some cases, a treatment may comprise a drug for treating a condition (e.g. a condition as described herein). In some cases, the one or more treatments may comprise a pharmacological treatment, a surgical treatment, a procedure, or any combination thereof. Methods for treating one or more conditions are described in International Application Nos. PCT/US2023/020752, and PCT/US2024/010125, each of which is incorporated herein by reference in its entirety for any purpose.

The one or more treatments of the methods described herein may comprise a treatment useful for the treatment of an autoimmune condition (e.g. lupus). In some cases, the one or more treatments may comprise a cereblon E3 ligase modulator. The cereblon E3 ligase modulator may bind to a Cereblon protein. The cereblon E3 ligase modulator may label proteins with ubiquitin for targeted degradation (e.g. by a proteosome). The cereblon E3 ligase modulator may comprise thalidomide, lenalidomide, pomalidomide, mezigdomide, iberdomide, golcadomide, an analogue thereof, or any combination thereof. In some cases, the cereblon E3 ligase modulator may comprise iberdomide.

The one or more treatments described herein may be formulated in a variety of ways. In some cases, the one or more treatments may be formulated as tablets, capsules, injectables, IV fluids, or any combination thereof. In some cases, the treatment may be formulated into doses (e.g. a first dose, a second dose and/or an initial dose). The dose of the one or more treatments (e.g. the cereblon E3 ligase modulator) may be based on the weight of the subject described herein. In some cases, a dose of a cereblon E3 ligase modulator (e.g. iberdomide) may be at least about 0.05 milligrams (mg) cereblon E3 ligase modulator/kilogram (kg) of the subject, at least about 0.1 mg cereblon E3 ligase modulator/kg of the subject, at least about 0.2 mg cereblon E3 ligase modulator/kg of the subject, at least about 0.3 mg cereblon E3 ligase modulator/kg of the subject, at least about 0.4 mg cereblon E3 ligase modulator/kg of the subject, at least about 0.5 mg cereblon E3 ligase modulator/kg of the subject, at least about 0.6 mg cereblon E3 ligase modulator/kg of the subject, at least about 0.7 mg cereblon E3 ligase modulator/kg of the subject, at least about 0.8 mg cereblon E3 ligase modulator/kg of the subject, at least about 0.9 mg cereblon E3 ligase modulator/kg of the subject, at least about 1 mg cereblon E3 ligase modulator/kg of the subject, at least about 1.5 mg cereblon E3 ligase modulator/kg of the subject, at least about 2 mg cereblon E3 ligase modulator/kg of the subject, at least about 3 mg cereblon E3 ligase modulator/kg of the subject, at least about 4 mg cereblon E3 ligase modulator/kg of the subject, at least about 5 mg cereblon E3 ligase modulator/kg of the subject, at least about 6 mg cereblon E3 ligase modulator/kg of the subject, at least about 7 mg cereblon E3 ligase modulator/kg of the subject, at least about 8 mg cereblon E3 ligase modulator/kg of the subject, at least about 9 mg cereblon E3 ligase modulator/kg of the subject, at least about 10 mg cereblon E3 ligase modulator/kg of the subject, at least about 15 mg cereblon E3 ligase modulator/kg of the subject, at least about 20 mg cereblon E3 ligase modulator/kg of the subject, at least about 30 mg cereblon E3 ligase modulator/kg of the subject, or higher. In some cases, a dose of a cereblon E3 ligase modulator (e.g. iberdomide) may be at most about 0.05 milligrams (mg) cereblon E3 ligase modulator/kilogram (kg) of the subject, at most about 0.1 mg cereblon E3 ligase modulator/kg of the subject, at most about 0.2 mg cereblon E3 ligase modulator/kg of the subject, at most about 0.3 mg cereblon E3 ligase modulator/kg of the subject, at most about 0.4 mg cereblon E3 ligase modulator/kg of the subject, at most about 0.5 mg cereblon E3 ligase modulator/kg of the subject, at most about 0.6 mg cereblon E3 ligase modulator/kg of the subject, at most about 0.7 mg cereblon E3 ligase modulator/kg of the subject, at most about 0.8 mg cereblon E3 ligase modulator/kg of the subject, at most about 0.9 mg cereblon E3 ligase modulator/kg of the subject, at most about 1 mg cereblon E3 ligase modulator/kg of the subject, at most about 1.5 mg cereblon E3 ligase modulator/kg of the subject, at most about 2 mg cereblon E3 ligase modulator/kg of the subject, at most about 3 mg cereblon E3 ligase modulator/kg of the subject, at most about 4 mg cereblon E3 ligase modulator/kg of the subject, at most about 5 mg cereblon E3 ligase modulator/kg of the subject, at most about 6 mg cereblon E3 ligase modulator/kg of the subject, at most about 7 mg cereblon E3 ligase modulator/kg of the subject, at most about 8 mg cereblon E3 ligase modulator/kg of the subject, at most about 9 mg cereblon E3 ligase modulator/kg of the subject, at most about 10 mg cereblon E3 ligase modulator/kg of the subject, at most about 15 mg cereblon E3 ligase modulator/kg of the subject, at most about 20 mg cereblon E3 ligase modulator/kg of the subject, at most about 30 mg cereblon E3 ligase modulator/kg of the subject, or lower. In some cases, a dose of a cereblon E3 ligase modulator (e.g. iberdomide) may be about 0.05 mg cereblon E3 ligase modulator/kg of the subject-30 mg cereblon E3 ligase modulator/kg of the subject, about 0.1 mg cereblon E3 ligase modulator/kg of the subject-20 mg cereblon E3 ligase modulator/kg of the subject, about 0.2 mg cereblon E3 ligase modulator/kg of the subject-15 mg cereblon E3 ligase modulator/kg of the subject, about 0.3 mg cereblon E3 ligase modulator/kg of the subject-10 mg cereblon E3 ligase modulator/kg of the subject, about 0.4 mg cereblon E3 ligase modulator/kg of the subject-9 mg cereblon E3 ligase modulator/kg of the subject, about 0.5 mg cereblon E3 ligase modulator/kg of the subject-8 mg cereblon E3 ligase modulator/kg of the subject, about 0.6 mg cereblon E3 ligase modulator/kg of the subject-7 mg cereblon E3 ligase modulator/kg of the subject, about 0.7 mg cereblon E3 ligase modulator/kg of the subject-6 mg cereblon E3 ligase modulator/kg of the subject, about 0.8 mg cereblon E3 ligase modulator/kg of the subject-5 mg cereblon E3 ligase modulator/kg of the subject, about 0.9 mg cereblon E3 ligase modulator/kg of the subject-4 mg cereblon E3 ligase modulator/kg of the subject, about 1 mg cereblon E3 ligase modulator/kg of the subject-3 mg cereblon E3 ligase modulator/kg of the subject, or about 1.5 mg cereblon E3 ligase modulator/kg of the subject-2 mg cereblon E3 ligase modulator/kg of the subject.

The dose of the one or more treatments (e.g. cereblon E3 ligase modulator) may be based on the weight of the one or more treatments. A dose of the cereblon E3 ligase modulator (e.g. iberdomide) may be at least about 0.5 mg, at least about 1 mg, at least about 5 mg, at least about 10 mg, at least about 20 mg, at least about 25 mg, at least about 30 mg, at least about 40 mg, at least about 50 mg, at least about 60 mg, at least about 70 mg, at least about 80 mg, at least about 90 mg, at least about 100 mg, at least about 150 mg, at least about 200 mg, at least about 250 mg, at least about 300 mg, at least about 350 mg, at least about 400 mg, or higher. A dose of the cereblon E3 ligase modulator (e.g. iberdomide) may be at most about 0.5 mg, at most about 1 mg, at most about 5 mg, at most about 10 mg, at most about 20 mg, at most about 25 mg, at most about 30 mg, at most about 40 mg, at most about 50 mg, at most about 60 mg, at most about 70 mg, at most about 80 mg, at most about 90 mg, at most about 100 mg, at most about 150 mg, at most about 200 mg, at most about 250 mg, at most about 300 mg, at most about 350 mg, at most about 400 mg, or higher. A dose of the cereblon E3 ligase modulator (e.g. iberdomide) may be about 0.5-400 mg, about 1-350 mg, about 5-300 mg, about 10-250 mg, about 20-200 mg, about 25-150 mg, about 30-100 mg, about 40-90 mg, about 50-80 mg, or about 60-70 mg.

In some cases, the cereblon E3 ligase modulator treatments may comprise one or more additional components. The one or more additional components may be other drugs for treating a condition. In some cases, the one or more additional components may be other drugs for treating an autoimmune condition (e.g. lupus). For example, the one or more additional components may comprise one or more nonsteroidal anti-inflammatory drugs (NSAIDs), one or more corticosteroids, one or more immunosuppressants, one or more biologics, one or more antimalarial drugs, or a combination thereof. The one or more NSAIDs may comprise Advil, Motrin IB, or a combination thereof. The one or more corticosteroids may comprise prednisone, methylprednisolone, dexamethasone, hydrocortisone, or any combination thereof. In some cases, the one or more immunosuppressants may comprise azathioprine (e.g. Imuran and Azasan), mycophenolate (e.g. Cellcept), methotrexate (e.g. Trexall and Xatmep), cyclosporine (e.g. Sandimmune, Neoral, Gengraf) and leflunomide (e.g. Arava) The one or more biologis may comprise belimumab (e.g. Benlysta), Rituximab (e.g. Rituxan), or any combination thereof. The one or more antimalarial drugs may comprise hydroxychloroquine (e.g. Plaquenil and Sovuna). In some cases, the one or more additional components may be used for treating one or more symptoms associated with an autoimmune condition (e.g. lupus). In some cases, the one or more additional components may be useful for formulating the cereblon E3 ligase modulator (e.g. iberdomide). For example, the one or more additional components may comprise one or more fillers (e.g. lactose, and/or mannitol), one or more binders (e.g. starch), one or more lubricants (e.g. talc), one or more coating agents (e.g. sucrose, cellulose acetate phthalate, or a combination thereof), one or more preservatives, or a combination thereof. In some cases, the one or more additional components may comprise one or more GLP-1 agonists. The one or more GLP-1 agonists may comprise semaglutide, liraglutide, dulaglutide, exenatide, tirzepatide, orforglipron, or any combination thereof.

The methods described herein may involve providing a treatment recommendation. The treatment recommendation may comprise dosing information, timing information, or a combination thereof. In some cases, a given dose may be recommended to a subject for one or more courses. For example, a treatment recommendation may comprise a treatment course involving a single dose, at least 2 doses, at least 3 doses, at least 4 doses, at least 5 doses, at least 6 doses, at least 7 doses, at least 8 doses, at least 9 doses, at least 10 doses, or more. In some cases, the treatment recommendation may comprise a recommendation to administer a dose at least every 1 day, at least every 2 days, at least every 3 days, at least every 4 days, at least every 5 days, at least every 6 days, at least every 7 days, at least every 8 days, at least every 9 days, at least every 10 days, at least every 1 week, at least every 2 weeks, at least every 3 weeks, at least every 4 weeks, at least every 5 weeks, at least every 8 weeks, at least every 10 weeks, at least every 12 weeks, or more.

The methods described herein may involve administering one or more doses of a treatment. The administering of the one or more doses of a treatment (e.g. iberdomide) may involve administering a single dose, at least 2 doses, at least 3 doses, at least 4 doses, at least 5 doses, at least 6 doses, at least 7 doses, at least 8 doses, at least 9 doses, at least 10 doses, or more. In some cases, administering a dose of the treatment may take place at least every 1 day, at least every 2 days, at least every 3 days, at least every 4 days, at least every 5 days, at least every 6 days, at least every 7 days, at least every 8 days, at least every 9 days, at least every 10 days, at least every 1 week, at least every 2 weeks, at least every 3 weeks, at least every 4 weeks, at least every 5 weeks, at least every 8 weeks, at least every 10 weeks, at least every 12 weeks, or more.

### Gene Sets

The methods described herein may comprise one or more gene sets. The one or more gene sets may be used for determining one or more endotypes as described herein. The one or more gene sets may comprise collections of genes with similar function and/or biological significance. For example, a gen set of the one or more gene sets as described herein may comprise genes associated with identification of a cell type, analysis of a biological pathway (e.g. a signaling pathway), identification of a treatment response, expression of one or more proteins, or any combination thereof. A gene set of the one or more gene sets may comprise genes that may demonstrate increased expression, decreased expression, no expression, or a combination thereof in the context of a disease state. For example, a gene set may comprise genes for identifying B cells in a sample. The genes for identifying B cells in a sample may demonstrate increased expression in inflammatory diseases.

The gene sets described herein may be used for determining and/or identifying one or more endotypes, as described herein. The gene sets may comprise a variety of genes. In some cases, the one or more gene sets may comprise the same number of genes, different number of genes, or a combination thereof. In some cases, a gene set may comprise at least about 1 gene, at least about 2 genes, at least about 3 genes, at least about 4 genes, at least about 5 genes, at least about 6 genes, at least about 7 genes, at least about 8 genes, at least about 9 genes, at least about 10 genes, at least about 11 genes, at least about 12 genes, at least about 13 genes, at least about 14 genes, at least about 15 genes, at least about 20 genes, at least about 25 genes, at least about 30 genes, at least about 35 genes, at least about 40 genes, at least about 50 genes, at least about 60 genes, at least about 70 genes, at least about 80 genes, at least about 90 genes, at least about 100 genes, at least about 125 genes, at least about 150 genes, at least about 175 genes, at least about 200 genes, or more genes. In some cases, a gene set may comprise at most about 1 gene, at most about 2 genes, at most about 3 genes, at most about 4 genes, at most about 5 genes, at most about 6 genes, at most about 7 genes, at most about 8 genes, at most about 9 genes, at most about 10 genes, at most about 11 genes, at most about 12 genes, at most about 13 genes, at most about 14 genes, at most about 15 genes, at most about 20 genes, at most about 25 genes, at most about 30 genes, at most about 35 genes, at most about 40 genes, at most about 50 genes, at most about 60 genes, at most about 70 genes, at most about 80 genes, at most about 90 genes, at most about 100 genes, at most about 125 genes, at most about 150 genes, at most about 175 genes, at most about 200 genes, or fewer genes. In some cases, a gene set may comprise about 1-200 genes, about 2-175 genes, about 3-150 genes, about 4-125 genes, about 5-100 genes, about 6-90 genes, about 7-80 genes, about 8-70 genes, about 9-60 genes, about 10-50 genes, about 11-40 genes, about 12-35 genes, about 13-30 genes, about 14-25 genes, or about 15-20 genes.

The one or more gene sets as described herein may comprise one or more gene sets of Table 1. For example, in some cases one or more endotypes may be determined and/or identified based on analysis of a plurality of gene sets. The plurality of gene sets may comprise all of the gene sets of Table 1, a subset of gene sets from Table 1, or none of the gene sets from table 1. In some cases, the plurality of gene sets may comprise at least about 1 gene set, at least about 2 gene sets, at least about 3 gene sets, at least about 4 gene sets, at least about 5 gene sets, at least about 6 gene sets, at least about 7 gene sets, at least about 8 gene sets, at least about 9 gene sets, at least about 10 gene sets, at least about 11 gene sets, at least about 12 gene sets, at least about 13 gene sets, at least about 14 gene sets, at least about 15 gene sets, at least about 16 gene sets, at least about 17 gene sets, at least about 18 gene sets, at least about 19 gene sets, at least about 20 gene sets, at least about 21 gene sets, at least about 22 gene sets, at least about 23 gene sets, at least about 24 gene sets, at least about 25 gene sets, at least about 26 gene sets, at least about 27 gene sets, at least about 28 gene sets, at least about 29 gene sets, at least about 30 gene sets, at least about 31 gene sets, at least about 32 gene sets, at least about 33 gene sets, at least about 34 gene sets, at least about 35 gene sets, at least about 36 gene sets, at least about 37 gene sets, at least about 38 gene sets, at least about 39 gene sets, at least about 40 gene sets, at least about 41 gene sets, at least about 42 gene sets, at least about 43 gene sets, at least about 44 gene sets, at least about 45 gene sets, at least about 46 gene sets, at least about 47 gene sets, or 48 gene sets from Table 1. In some cases, the plurality of gene sets may comprise at most about 1 gene set, at most about 2 gene sets, at most about 3 gene sets, at most about 4 gene sets, at most about 5 gene sets, at most about 6 gene sets, at most about 7 gene sets, at most about 8 gene sets, at most about 9 gene sets, at most about 10 gene sets, at most about 11 gene sets, at most about 12 gene sets, at most about 13 gene sets, at most about 14 gene sets, at most about 15 gene sets, at most about 16 gene sets, at most about 17 gene sets, at most about 18 gene sets, at most about 19 gene sets, at most about 20 gene sets, at most about 21 gene sets, at most about 22 gene sets, at most about 23 gene sets, at most about 24 gene sets, at most about 25 gene sets, at most about 26 gene sets, at most about 27 gene sets, at most about 28 gene sets, at most about 29 gene sets, at most about 30 gene sets, at most about 31 gene sets, at most about 32 gene sets, at most about 33 gene sets, at most about 34 gene sets, at most about 35 gene sets, at most about 36 gene sets, at most about 37 gene sets, at most about 38 gene sets, at most about 39 gene sets, at most about 40 gene sets, at most about 41 gene sets, at most about 42 gene sets, at most about 43 gene sets, at most about 44 gene sets, at most about 45 gene sets, at most about 46 gene sets, or at most about 47 gene sets from Table 1. In some cases, the plurality of gene sets may comprise about 1-48 gene sets, about 2-47 gene sets, about 3-46 gene sets, about 4-45 gene sets, about 5-44 gene sets, about 6-43 gene sets, about 7-42 gene sets, about 8-41 gene sets, about 9-40 gene sets, about 10-39 gene sets, about 11-38 gene sets, about 12-37 gene sets, about 13-36 gene sets, about 14-35 gene sets, about 15-34 gene sets, about 16-33 gene sets, about 17-32 gene sets, about 18-31 gene sets, about 19-30 gene sets, about 20-29 gene sets, about 21-28 gene sets, about 22-27 gene sets, about 23-26 gene sets, or about 24-25 gene sets from Table 1.

In some cases, a gene set of the plurality of gene sets described herein may comprise a subset of genes listed in Table 1. For example, a gene set may comprise a percentage of genes listed in a gene set in Table 1. In some cases, a gene set of the plurality of gene sets described herein may comprise at least about 5% of genes listed in a gene set in Table 1, at least about 10% of genes listed in a gene set in Table 1, at least about 15% of genes listed in a gene set in Table 1, at least about 20% of genes listed in a gene set in Table 1, at least about 25% of genes listed in a gene set in Table 1, at least about 30% of genes listed in a gene set in Table 1, at least about 35% of genes listed in a gene set in Table 1, at least about 40% of genes listed in a gene set in Table 1, at least about 45% of genes listed in a gene set in Table 1, at least about 50% of genes listed in a gene set in Table 1, at least about 55% of genes listed in a gene set in Table 1, at least about 60% of genes listed in a gene set in Table 1, at least about 65% of genes listed in a gene set in Table 1, at least about 70% of genes listed in a gene set in Table 1, at least about 75% of genes listed in a gene set in Table 1, at least about 80% of genes listed in a gene set in Table 1, at least about 85% of genes listed in a gene set in Table 1, at least about 90% of genes listed in a gene set in Table 1, at least about 95% of genes listed in a gene set in Table 1, or about 100% of genes listed in a gene set in Table 1. In some cases, a gene set of the plurality of gene sets described herein may comprise at most about 5% of genes listed in a gene set in Table 1, at most about 10% of genes listed in a gene set in Table 1, at most about 15% of genes listed in a gene set in Table 1, at most about 20% of genes listed in a gene set in Table 1, at most about 25% of genes listed in a gene set in Table 1, at most about 30% of genes listed in a gene set in Table 1, at most about 35% of genes listed in a gene set in Table 1, at most about 40% of genes listed in a gene set in Table 1, at most about 45% of genes listed in a gene set in Table 1, at most about 50% of genes listed in a gene set in Table 1, at most about 55% of genes listed in a gene set in Table 1, at most about 60% of genes listed in a gene set in Table 1, at most about 65% of genes listed in a gene set in Table 1, at most about 70% of genes listed in a gene set in Table 1, at most about 75% of genes listed in a gene set in Table 1, at most about 80% of genes listed in a gene set in Table 1, at most about 85% of genes listed in a gene set in Table 1, at most about 90% of genes listed in a gene set in Table 1, at most about 95% of genes listed in a gene set in Table 1, or about 100% of genes listed in a gene set in Table 1. In some cases, a gene set of the plurality of gene sets described herein may comprise about 5%-100% of genes listed in a gene set in Table 1, about 10%-95% of genes listed in a gene set in Table 1, about 15%-90% of genes listed in a gene set in Table 1, about 20%-85% of genes listed in a gene set in Table 1, about 25%-80% of genes listed in a gene set in Table 1, about 30%-75% of genes listed in a gene set in Table 1, about 35%-70% of genes listed in a gene set in Table 1, about 40%-65% of genes listed in a gene set in Table 1, about 45%-60% of genes listed in a gene set in Table 1, or about 50%-55% of genes listed in a gene set in Table 1.

TABLE 1
contains descriptions and corresponding gene sets.

| Description | Gene set |
|---|---|
| B Cell signature genes | AICDA, BANK1, BLK, BLNK, CD19, CD22, CD79A, |
| | CD79B, CLEC17A, CR2, DAPP1, DTX1, FCRL1, FCRL2, |
| | FCRL3, FCRL4, FCRL5, HLA-DOB, IGHD, IGHM, LY6D, |
| | MS4A1, PAX5, POU2AF1, SH2B2, TNFRSF13B, |
| | TNFRSF13C, VPREB1, VPREB3, ZBTB32, ZNF318 |
| Endothelial Cell signature genes | DLC1, ECSCR, EMCN, FLT1, KDR, LDB2, LRRC32, |
| | MEIS2, PLAT, PTPRB, SELE, TM4SF1, TM4SF18, VWF |
| Erythrocyte signature genes 1 | BSG, GFI1B, GYPA, GYPB, GYPE, ICAM4, KEL, NFE2, |
| | RHD, SLC4A1, TRIM10, TSPO2 |
| Erythrocyte signature genes 2 | BSG, GFI1B, GYPA, GYPB, GYPE, ICAM4, KEL, NFE2, |
| | RHD, SLC4A1, TRIM10, TSPO2 |
| Fibroblast signature genes | 43894, ADAM33, ADAMTS6, AGTR1, ALPK2, ANGPTL2, |
| | ANKRD45, ANO2, ANPEP, ARMC9, ASPN, BDKRB2, |
| | BDNF, BMPER, C11orf87, C1R, C1S, CATSPER3, |
| | CCDC102B, CCDC80, CCDC81, CEMIP, CHAC1, CHRM2, |
| | CLMP, CNN1, COL14A1, COL3A1, COL5A1, COLEC10, |
| | CPXM2, CPZ, CRABP2, CXCL12, DCN, DDR2, DKK1, |
| | DMRTA1, EGFL6, ELOVL2, EMILIN1, FAM180A, FBLN7, |
| | FBN1, FGF5, FMN2, FOXF2, FST, FSTL1, GFRA1, GLIS1, |
| | GLT8D2, GPR176, GREM1, GREM2, GRIK2, GUCY1A2, |
| | HSD17B2, HSPA2, HSPB3, IL19, IQCD, KCNMB2, |
| | KIRREL3, KRT34, KRTAP1-5, KRTAP3-1, L1CAM, LAYN, |
| | LMOD1, LOXL4, LUM, LY6K, MFAP4, MFAP5, MGARP, |
| | MGP, MKX, MMP2, MXRA5, MXRA8, MYPN, NEXN, |
| | NFASC, NID2, NTF3, OLFML3, P3H3, P4HA3, PAMR1, |
| | PAX3, PCDHGA2, PCDHGA3, PCDHGA7, PDE8B, |
| | PDGFRA, PDGFRL, PDZRN3, PLA2R1, PLEKHA4, PLPP4, |
| | PLPPR4, PRKG1, PSG5, PTPRQ, PTRF, SEMA3A, |
| | SEMA5A, SEMA6D, SHOX, SLC16A2, SMIM2, SPARCL1, |
| | SPHKAP, SSTR1, STC1, STXBP6, SUSD5, SVEP1, TBX15, |
| | THBS2, TIMP2, TIMP3, TMEM119, TMEM130, TMEM47, |
| | TRHDE, TRPC4, UACA, UBL4B, VAT1L, VEGFC, |
| | WNT5A, WNT5B, ZFPM2 |
| GC B Cell signature genes | FCRLA, GCSAM, KLHL6, LRMP, NUGGC, RGS13 |
| Granulocyte signature genes | CLC, HSH2D, MS4A2, PGLYRP1, PRG2, SYNE1 |
| Keratinocyte signature genes | ABLIM, AKR1C1, ALDH8, ALOX12B, ANXA8, AQP3, |
| | ATDC, BPAG1, CA12, CCND2, CD24, CDH3, CDKN1A, |
| | CDSN, COL17A1, CST6, CSTA, DD96, DSC1, DSG1, DSP, |
| | EGFR, EVPL, FLG, G0S2, GJA1, GLUL, GNA15, HBP17, |
| | IFI27, IFITM1, IGFBP7, ITGA3, ITGB4, IVL, JUNB, JUP, |
| | KLK11, KLK7, KLK8, KRT1, KRT14, KRT15, KRT16, |
| | KRT2A, KRT5, KRT6A, LAMA3, LGALS7, LOR, NOTCH3, |
| | PPL, PRDX2, PRSS11, PRSS2, PRSS4, S100A2, SERPINB2, |
| | SERPINB3, SERPINB4, SERPINB5, SERPINB7, SERPINE1, |
| | SFN, SPINK5, SPRR1B, TACSTD2, TFAP2A, TGM1, TP63, |
| | TUBA1, XP5 |
| Langerhans Cell signature genes | CD1B, CD1C, CD1E, CD207 |
| LDG signature genes | AZU1, CAMP, CEACAM3, CEACAM4, CEACAM6, |
| | CEACAM8, CTSG, DEFA4, ELANE, MPO, OLFM4, |
| | RNASE3 |
| Melanocyte signature genes | ASIP, CITED1, DCT, GPNMB, GPR143, MITF, MLANA, |
| | MLPH, OCA2, PMEL, SLC24A5, SLC45A2, TYR, TYRP1 |
| Monocyte signature genes | ADGRE1, C1QA, C1QB, C1QC, C2, CD14, CD300C, |
| | CD300E, CD5L, CD68, CLEC5A, CSF1R, CYBB, FOLR2, |
| | LILRA1, MARCO, MERTK, MS4A7, MSR1, SPIC |
| Monocyte/Myeloid Cell signature | ADGRE2, ADGRE3, AIF1, APOC1, BPI, BST1, C4A, C4B, |
| genes | C4BPA, C4BPB, C5, C6, C8A, C9, CD163, CD1D, CD209, |
| | CD300LF, CD33, CFD, CFP, CHIT1, CLEC12A, CLEC12B, |
| | CLEC1A, CLEC4A, CLEC4D, CLEC4E, CLEC4G, CLEC6A, |
| | CLEC7A, CRISP3, CSF2RA, CSF2RB, CST3, CTSS, F12, |
| | FCER1A, FCER1G, FCGR1A, FCGR1B, FCGR2A, FCGR2C, |
| | FLT3, GRN, IGSF6, ITGAX, LGALS12, LGALS4, LGALS9, |
| | LILRA2, LILRA5, LILRA6, LILRB2, LY86, LYVE1, LYZ, |
| | MEFV, MMP8, MNDA, MPEG1, MS4A4A, MS4A6A, |
| | NLRP12, NLRP3, NOD2, OLR1, OSCAR, OSM, PILRA, |
| | PRAM1, RETN, S100A12, S100A8, S100A9, SCARB1, |
| | SECTM1, SEMA4A, SERPING1, SGK1, SIGLEC1, |
| | SIGLEC10, SIGLEC14, SIGLEC5, SLC11A1, SLITRK4, |
| | SMPDL3B, SPI1, TEK, THBD, TLR2, TLR8, TNFSF13B, |
| | TREM1, TREML4, TYROBP, VENTX, VSIG4, VSTM1 |
| Neutrophil signature genes | ARG1, BMX, CD177, CSF3R, DEFA1, DEFA1B, DEFA3, |
| | DEFB103A, DEFB103B, DEFB106B, DEFB136, DEFB4A, |
| | FPR2, OR1J2, PRTN3, SLC2A3, SLPI |
| NK Cell signature genes | KIR2DL4, KLRC3, KLRF1, NCAM1, NCR1, SH2D1B, |
| | TNFSF11, TXK |
| pDC signature genes | CLEC4C, IRF7, LILRA4, NRP1, PACSIN1, PLA2G5, |
| | PLAC8, PTCRA, SERPINF1, SLC15A4, TCF4 |
| Plasma Cell signature genes | CD38, CRELD2, ELL2, FKBP11, IGKV4-1, IGLV2-14, |
| | ITM2C, JCHAIN, MANF, MZB1, PDIA4, PRDX4, SDF2L1, |
| | SPATS2, TNFRSF17, UAP1 |
| Platelet signature genes | GP1BA, GP5, GP6, GP9, PF4, PF4V1, PLEK, PPBP, |
| | SLC35D3 |
| Skin-specific DC signature genes | CLEC10A, CLEC9A, GPR31, MRC1, XCR1 |
| T- Cell signature genes | BCL11B, CAMK4, CD28, CD3D, CD3G, CD5, CD6, |
| | GPR171, ITK, RGCC, TESPA1, THEMIS, TRAT1 |
| Th17 signature genes | CCR6, CXCR3, IL17A, IL17F, IL22, IL26, KLRB1, RORA, |
| | RORC |
| AA Metabolism signature genes | AASS, AGXT, AGXT2, ALDH4A1, ALDH7A1, ARG1, |
| | GAD1, GLUD1, GOT1, GPT2, GRHPR, IVD, MCCC2, OAT, |
| | OTC, OXCT1, PC, PSAT1, SDS, IL1RN, SOCS3, TNFAIP3 |
| Apoptosis signature genes | AIFM1, BAD, BAK1, BAX, BID, CASP10, CASP3, CASP6, |
| | CASP7, CASP8, CASP9, ENDOG, FAS, HTRA2, TNFRSF1A |
| Cell Cycle signature genes | ASPM, AURKA, AURKB, BRCA1, CCNB1, CCNB2, |
| | CCNE1, CDC20, CENPM, CEP55, E2F3, GINS2, MCM10, |
| | MCM2, MKI67, NCAPG, NDC80, PTTG1, TYMS |
| Complement Proteins signature | C1QA, C1QB, C1QC, C1R, C1S, C2, C3, C4A, C4B, C5, C6, |
| genes | C7, C8A, C8B, C8G, C9 |
| FAAO signature genes 1 | HAAO, HACL1, PEX13, PHYH, SLC27A2 |
| FABO signature genes 2 | ABCD1, ABCD2, ABCD3, ACAA2, ACACB, ACAD11, |
| | ACADL, ACADM, ACADS, ACADVL, ACAT1, ACAT2, |
| | ACOX1, ACOX2, ACOX3, ACOXL, ACSBG2, ACSL5, |
| | ADIPOQ, AKT2, AUH, BDH2, CPT1A, CPT2, CROT, |
| | DECR1, ECHDC1, ECHDC2, ECHS1, ECI1, ECI2, |
| | EHHADH, ETFA, ETFB, ETFDH, FABP1, GCDH, HADH, |
| | HADHA, HADHB, HIBCH, HSD17B4, IRS1, IRS2, IVD, |
| | LEP, PEX2, PEX5, PEX7, SESN2, SLC25A17, SLC27A2, |
| | TWIST1 |
| Glycolysis signature genes | ALDOA, ALDOB, ALDOC, ENO1, ENO2, ENO3, GAPDH, |
| | GCK, GPI, HK1, HK2, HK3, HKDC1, LDHA, LDHAL6A, |
| | LDHAL6B, LDHB, LDHC, PFKFB1, PFKFB2, PFKFB3, |
| | PFKFB4, PFKL, PFKM, PFKP, PGAM1, PGAM2, PGK1, |
| | PGK2, PKM, SLC2A1, SLC2A3, SLC2A4, TPI1 |
| IFN signature genes | EIF2AK2, GBP1, GBP2, GBP4, HERC5, HERC6, IFI27, |
| | IFI30, IFI35, IFI44, IFI44L, IFI6, IFIT1, IFIT2, IFIT3, |
| | IFIT5, IFITM1, IFITM2, IFITM3, ISG15, ISG20, MX1, MX2, |
| | OAS1, OAS2, OAS3, OASL, RSAD2, SAMD9, SAMD9L, |
| | SP100, SP110 |
| IL1 Cytokines signature genes | IL18, IL1B |
| IL12 Complex signature genes | IL12A, IL12B, IL12RB1, IL12RB2 |
| IL12 signature genes | ACLY, AKAP10, APOL3, BACH2, BRCA2, CALD1, CASK, |
| | CASP1, CCR5, CDKN3, CXCL10, CXCL9, CYBB, DEFA1, |
| | ETAA1, FASLG, FBXL2, FCER2, FCGR1A, GBP1, GBP2, |
| | GLS, GNPDA1, GSTM5, GZMB, HHEX, HP, HSPA6, IFNG, |
| | IL16, IL18BP, IL18R1, IL1A, INPP5D, INSIG1, IRF1, KLF2, |
| | KRT8, LIMK1, LINC00597, LY75, MMP25, NIN, NLRP1, |
| | PCDH9, SELL, SERPIND1, SLAMF1, SOCS1, STAT1, |
| | TAP2, TBX21, TFF1, TNFAIP2, TNFAIP3, TNFSF10, TXK |
| IL17 Complex signature genes | IL17A, IL17F, IL17RA, IL17RC, TRAF3, TRAF3IP2 |
| IL21 Complex signature genes | IL21, IL21R, IL2RG |
| IL23 Complex signature genes | IL12B, IL12RB1, IL23A, IL23R |
| Immunoproteasome signature genes | PSMB10, PSMB8, PSMB9 |
| Inflammasome signature genes | AIM2, CASP1, CASP5, CTSB, GSDMB, GSDMD, NAIP, |
| | NEK7, NLRC4, NLRP1, NLRP3, NOD2, P2RX7, PANX1, |
| | PYCARD, RIPK1 |
| OXPHOS signature genes | ATP5A1, ATP5B, ATP5D, ATP5E, ATP5F1, ATP5G1, |
| | ATP5G2, ATP5G3, ATP5H, ATP5I, ATP5J, ATP5J2, ATP5L, |
| | ATP5O, ATP5S, BCS1L, CEP89, COA1, COA3, COA4, |
| | COA5, COA6, COA7, COX10, COX10-AS1, COX11, |
| | COX14, COX15, COX16, COX17, COX18, COX19, COX20, |
| | COX4I1, COX4I2, COX5A, COX5B, COX6A1, COX6A2, |
| | COX6B1, COX6B2, COX6C, COX7A1, COX7A2, |
| | COX7A2L, COX7B, COX7B2, COX7C, COX8A, COX8C, |
| | CYC1, CYCS, DNAJC15, MT-ATP6, MT-ATP8, MT-CO1, |
| | MT-CO2, MT-CO3, MT-CYB, MT-ND1, MT-ND2, MT-ND3, |
| | MT-ND4, MT-ND4L, MT-ND5, MT-ND6, NDUFA1, |
| | NDUFA10, NDUFA11, NDUFA12, NDUFA13, NDUFA2, |
| | NDUFA3, NDUFA4, NDUFA4L2, NDUFA5, NDUFA6, |
| | NDUFA7, NDUFA8, NDUFA9, NDUFAB1, NDUFAF1, |
| | NDUFAF2, NDUFAF3, NDUFAF4, NDUFAF5, NDUFAF6, |
| | NDUFAF7, NDUFAF8, NDUFB1, NDUFB10, NDUFB11, |
| | NDUFB2, NDUFB2-AS1, NDUFB3, NDUFB4, NDUFB5, |
| | NDUFB6, NDUFB7, NDUFB8, NDUFB9, NDUFC1, |
| | NDUFC2, NDUFS1, NDUFS2, NDUFS3, NDUFS4, |
| | NDUFS5, NDUFS6, NDUFS7, NDUFS8, NDUFV1, |
| | NDUFV2, NDUFV3, NUBPL, OXA1L, RFESD, SCO1, |
| | SCO2, SLC25A4, SURF1, TACO1, TIMMDC1, TMEM126B, |
| | TRAP1, TTC19, UQCC1, UQCC2, UQCC3, UQCR10, |
| | UQCR11, UQCRB, UQCRC1, UQCRC2, UQCRFS1, |
| | UQCRH, UQCRHL, UQCRQ |
| Pentose Phosphate signature genes | G6PD, H6PD, PGD, PRPS1, PRPS1L1, PRPS2, RBKS, RGN, |
| | RPE, RPIA, TALDO1, TKT, TKTL1, TKTL2 |
| Peroxisome signature genes | ABCD3, ACAA1, ACOX1, ACOX2, ACOX3, CAT, DDO, |
| | DECR2, EHHADH, HAO2, HMGCL, HSDL2, ISOC1, KXD1, |
| | PAOX, PEX1, PEX10, PEX11A, PEX12, PEX16, PEX19, |
| | PEX26, PEX3, PEX5, PEX6, PEX7, PHYH, PIPOX, PMVK, |
| | PXMP2, SCP2, SLC25A17 |
| Proteasome signature genes | ADRM1, NGLY1, PAAF1, POMP, PSMA1, PSMA2, PSMA3, |
| | PSMA3-AS1, PSMA4, PSMA5, PSMA6, PSMA7, PSMA8, |
| | PSMB1, PSMB11, PSMB2, PSMB3, PSMB4, PSMB5, |
| | PSMB6, PSMB7, PSMC1, PSMC2, PSMC3, PSMC4, PSMC5, |
| | PSMC6, PSMD1, PSMD10, PSMD11, PSMD12, PSMD13, |
| | PSMD14, PSMD2, PSMD3, PSMD4, PSMD5, PSMD5-AS1, |
| | PSMD6, PSMD6-AS2, PSMD7, PSMD8, PSMD9, PSME1, |
| | PSME2, PSME3, PSME4, PSMF1, PSMG1, PSMG2, PSMG3, |
| | PSMG3-AS1, PSMG4, RAD23B, SHFM1, UBLCP1, |
| | UBQLN1, UCHL5, ZFAND2A |
| ROS Production signature genes | GPX1, GPX3 |
| T Cell IL12 signature genes | CCL5, CXCR1, GZMH, IFNG, IL12RB2, IL1RL1, LIFR, |
| | TNFSF10, TNFSF13B |
| T Cell IL23 signature genes | CCL17, CCL20, CCL22, CCL7, CCR1, CSF2, CXCL2, |
| | CXCR5, IL17A, IL17F, IL17RC, IL1R1, IL23R, IL6, ITGA3, |
| | PDGFB, TNF |
| TCA cycle signature genes | ACO2, CS, DLAT, DLD, DLST, FH, GLUD1, IDH1, IDH2, |
| | IDH3A, IDH3B, IDH3G, MDH2, MPC1, MPC2, OGDH, |
| | OGDHL, PDHA1, PDHA2, PDHB, PDHX, PDK1, PDK2, |
| | PDK3, PDK4, PDP1, PDP2, PDPR, SDHA, SDHAF1, |
| | SDHAF2, SDHAF3, SDHAF4, SDHB, SDHC, SDHD, |
| | SUCLA2, SUCLG1, SUCLG2, SUGCT |
| TNF signature genes | ACLY, ACSL1, ADGRE2, AK3, AKAP10, AMPD3, APOL3, |
| | ARID3A, ARSE, ASAP1, B4GALT5, BCL2A1, BHLHE41, |
| | BHMT, BIRC3, BRCA1, CALD1, CASP1, CASP10, CCL15, |
| | CCL20, CCL23, CCL3L1, CD37, CD38, CD83, CDKN3, |
| | CKB, CR2, CTNND2, CXCL1, CXCL2, CXCL3, CXCL8, |
| | CYP27B1, DAB2, EBI3, EGR1, EGR2, EPB41, EREG, |
| | ETAA1, F3, FABP1, FBXL2, FCER2, FCGR2A, FLJ11129, |
| | FLNA, G0S2, GBP1, GCH1, GJB2, GLS, GMIP, GP1BA, |
| | GRK3, HCAR3, HHEX, HOMER2, HP, ICAM1, IDO1, IFI44, |
| | IKBKG, IL16, IL18, IL1A, IL1B, IL1RN, IL6, INHBA, |
| | INSIG1, ITGA6, KITLG, KLF1, KMO, LGALS3BP, |
| | MAP3K4, MARCKS, MGLL, MMP19, MN1, MRPS15, MSC, |
| | MTF1, MX1, NAMPT, NELL2, NFKB1, NFKB2, NFKBIA, |
| | NFKBIZ, NKX3-2, NR3C1, OAS3, PATJ, PDE4DIP, PDPN, |
| | PIAS4, PLAUR, PTGES, PTGS2, RELB, RPGR, RPS9, |
| | SDC4, SERPIND1, SFRP1, SH3BP5, SLAMF1, SLC30A4, |
| | SOD2, SPI1, SSPN, STAT4, TAF15, TAP2, TBX3, TFF1, |
| | TNF, TNFAIP2, TNFAIP3, TNFRSF11A, TRAF1, TSC22D1, |
| | TYROBP, UBE2C, VEGFA, WT1 |
| Unfolded Protein signature genes | B4GALT3, CALR, CALU, CANX, CDS2, CHST12, CHST2, |
| | DERL1, DERL2, DNAJC3, EDEM2, EDEM3, EMC9, |
| | ERAP1, ERGIC2, ERO1L, EXT1, GALNT2, GOLT1B, |
| | HERPUD1, HYOU1, IER3IP1, IMPAD1, KDELC1, |
| | KDELR2, LMAN2, LPGAT1, MAN1A1, MANEA, MANF, |
| | NUCB2, PDIA4, PDIA6, PIGK, PPIB, SEC24D, SEC61G, |
| | SPCS3, SSR1, SSR3, TRAM1, TRAM2, UGGT1, XBP1 |
| TGFB Fibroblast signature genes | ABTB2, ACOX1, ACTA2, ACTC, ACTN1, ACTN3, |
| | ADAM12, ADAM19, ADAMTS4, ADCY7, AK3, ALS2CR4, |
| | AMIGO2, ANGPTL4, AQP1, ARK5, ARL4A, ARNTL, ASE- |
| | 1, ASNS, ATOH8, ATP10A, ATP1B1, AVP, AXUD1, |
| | B4GALT1, BAG3, BFAR, BHLHB2, BLOC1S2, BM039, |
| | BMP6, BMPR2, C10orf22, C10orf30, C14orf138, C14orf31, |
| | C16orf30, C18orf1, C20orf139, C20orf39, C21orf93, |
| | C5orf13, C6orf145, C6orf85, C9orf19, C9orf3, C9orf62, |
| | CALM2, CARD4, CBFB, CCDC8, CCL2, |
| | CDH2, CDKN2B, CEBPA, CH25H, CHIC2, CHST11, |
| | CHST5, CHSY1, CLC, CMKOR1, CNN3, COL4A1, |
| | COL4A2, COL5A1, COL5A2, COMP, CREB3L2, CRLF1, |
| | CRY1, CSRP1, CSRP2, CTGF, CTPS, CXCL12, CXXC5, |
| | CYR61, DACT1, DDIT4, DLC1, DLX2, DNAJB4, DNAJB5, |
| | DNAJB9, DOK5L, DSP, DTR, DUSP1, DYRK2, E2F7, |
| | EIF4EBP1, ELN, ENC1, ENPP1, EPHB3, ERN1, EYA2, |
| | FBXO32, FGF18, FGF2, FGFR3, FGFRL1, FHL2, FLJ10350, |
| | FLJ10357, FLJ10378, FLJ14054, FLJ20364, FLJ20366, |
| | FLJ20701, FLJ22938, FLJ39370, FLJ45248, FN5, FOXP1, |
| | FSTL3, FUS, FZD8, GABRE, GADD45B, GARS, GAS7, |
| | GATA6, GDF15, GDF6, GEM, GLS, GNPNAT1, GOPC, |
| | GPAM, GPR68, GPT2, GSTT2, HCMOGT1, HES1, HIF1A, |
| | HILS1, HNRPAB, HNRPK, HOMER1, HOXB2, HOXC8, |
| | HSPA5, HSPB7, HSXIAPAF1, ID1, ID3, ID4, IER3, IER5L, |
| | IGF1, IL11, IL21R, IL4R, IL6, ITR, IVNS1ABP, JUNB, K- |
| | ALPHA-1, KCNE4, KCNG1, KCNK1, KCNN4, KCNS3, |
| | KCTD11, KIAA0033, KIAA0280, KIAA1102, KIAA1644, |
| | KIAA1754, KLF10, KLF13, KLF2, LDHA, LHFPL2, LIF, |
| | LIM, LIMK1, LIMK2, LIMS3, LMCD1, LMO4, LOC222171, |
| | LOC283824, LOC284454, LOC339047, LOC440502, |
| | LOC51333, LRIG1, LRRC8, LTBP2, MAP3K2, MBD4, |
| | MGC14376, MGC15476, MGC16121, MGC29875, |
| | MGC4504, MGC45871, MGC8685, MGLL, MICAL2, |
| | MICAL-L1, MIR100HG, MONDOA, MRC2, MSX1, |
| | MTCH1, MTHFD2, NEDD4, NEDD9, NET1, NFATC1, |
| | NFYC, NGEF, NID67, NKD2, NLF1, NNMT, NP, NPAS1, |
| | NPTX1, NRBF2, NRG1, NUP98, ODC1, P4HA2, P4HA3, |
| | PACSIN2, PAWR, PDGFA, PDLIM4, PFKP, PGK1, |
| | PGM2L1, PGM3, PHF17, PHLDA2, PHLDB1, PICALM, |
| | PIM1, PITX2, PKM2, PLAU, PLAUR, PLEKHA1, PLK3, |
| | PLOD2, PNMA1, PODXL, POFUT2, PPP1R13L, PPP1R14C, |
| | PPP1R3B, PRICKLE2, PRKAB2, PRO1855, PRPS1, |
| | PRPS1L1, PRRX2, PSAT1, PTDSR, PTPNS1, RAI14, RAI17, |
| | RASL11B, RGS3, RKHD3, RNF126, RPL21, RPL5, RTTN, |
| | RUNX1, RUNX2, RUSC2, S100A16, SAMD11, SARS, SCD, |
| | SCHIP1, SDFR1, SERP1, SERPINE1, SERTAD1, SERTAD4, |
| | SGCG, SGK, SH3MD1, SIAT4A, SKIL, SLC10A3, |
| | SLC16A3, SLC19A2, SLC1A5, SLC20A1, SLC26A1, |
| | SLC2A1, SLC38A5, SLC39A14, SLC4A2, SLC7A11, |
| | SLC7A5, SMAD7, SMARCB1, SNAI1, SNF1LK, SNX24, |
| | SOX4, SOX9, SPARC, SPHK1, SRF, STC2, STCH, STK38L, |
| | SYNJ2, SYVN1, TBX3, TD-60, TES, TGFB1, TGFBR1, |
| | TGM2, TIMP3, TIPARP, TMEPAI, TMPO, TNC, |
| | TNFRSF12A, TNFRSF19L, TPM1, TRIB1, TRIB2, TRIB3, |
| | TSK, TUBA3, TUBA6, TUBB2, TUBB3, TUBB4, TUBB6, |
| | TUFT1, UAP1, UCK2, UGDH, ULK1, UNC5B, UPP1, |
| | USP35, VEGF, VLDLR, VMP1, WNT5B, XBP1, ZNF281, |
| | ZNF336, ZNF469, ZNF537 |
| Anti-inflammation signature genes | IL1RN, SOCS3, TNFAIP3 |

### Samples

The methods described herein comprise one or more samples from a subject. The one or more samples form the subject may be provided before treatment, during treatment, after treatment, or a combination thereof. The one or more samples may be collected or have been collected form one or more time points. For example, a sample may from a time point before a subject begins treatment, a time point after a first treatment dose, a time point after a second treatment dose, a time point after a third treatment dose, or a combination thereof. The one or more samples may be used for determining whether a treatment is effective (e.g. whether a subject having lupus is responding to receiving one or more doses of a treatment). The one or more samples may be used for determining an initial dose of a treatment (e.g. determining whether a subject will be responsive to a treatment based, for example, on their endotype).

The one or more samples of the methods described herein may comprise a variety of sample types. In some cases, the one or more samples may comprise a body fluid sample, a tissue sample, an exogenous sample, or a combination thereof. The body fluid sample may comprise a urine sample, a saliva sample, a blood sample, a plasma sample, a whole blood sample, a vaginal secretion sample, a semen sample, a serum sample, a cerebrospinal fluid sample, a bone marrow sample, or a combination thereof. The tissue sample may comprise a biopsy, a surgical resection, a needle-core biopsy, a tissue slice, or a combination thereof. The exogenous sample may comprise a tissue culture sample, a cell culture sample, or a combination thereof. The blood sample may comprise plasma, buffy coat, erythrocytes, cfDNA, cell-free RNA (cfRNA), or a combination thereof. In some cases, the blood sample may comprise a whole blood sample. The whole blood sample may be processed by separating one or more components of whole blood. For example, a whole blood sample may be processed to separate a plasma layer, a buffy coat layer and a red blood cell layer of the blood sample. The plasma layer may be combined with a preservative for stabilization of RNA (e.g. stabilization of intracellular RNA). In some cases, the sample may be processed to purify mRNA from a blood sample or a processed blood sample. In some cases, a plasma layer from a processed blood sample may be lysed to break cell membranes. Cell lysis may be performed using guanidine isothiocyanate. In some cases, the sample may be treated with DNAse. Treating a sample with DNase may be used to remove DNA from the sample. In some cases, the sample may be treated to remove a subset of RNA (e.g. ribosomal RNA). A purification step may be performed to bind RNA from the sample (e.g. mRNA) to a silica membrane. The RNA from the sample may be release from the silica membrane into a buffer solution, water, or a combination thereof. The sample may comprise RNA (e.g. mRNA) from multiple cells within the sample (e.g. after cell lysis and processing). The sample may comprise cell-free RNA, intracellular RNA, or a combination thereof.

### Endotypes

The methods described herein may comprise using one or more endotypes, determining one or more endotypes, or a combination thereof. The one or more endotypes as described herein may be useful for classifying a subject as having a particular type of condition (e.g. lupus). For example, a sample from a subject may be analyzed and an endotype of the subject may be determined. The endotype may be useful in characterizing the type and/or severity of a condition of the subject. In some cases, an endotype of a subject may be used for treatment decisions and/or recommendations. For example, a subject may be determined to have an endotype and the endotype may be used to determine a treatment plan for the subject. In some cases, an endotype of the subject may be used for adjusting a treatment recommendation for a subject. An endotype of the methods described herein may be a subtype of a disease that is distinct from other subtypes of a disease based on underlying biological mechanisms or pathways. For example, a disease (e.g. lupus) may be heterogenous and comprise more than one endotype. The endotypes of the disease may be distinguished based on difference in their biological mechanisms or pathways. Methods for identifying, determining, and/or using one or more endotypes are described in International Application Nos. PCT/US2023/020752, and PCT/US2024/010125, each of which is incorporated herein by reference in its entirety for any purpose.

Endotypes may be based on a variety of biological information, including but not limited to gene expression data, protein expression data, metabolite expression data, clinical data, or a combination thereof. In some cases, a dataset comprising various biological information may be analyzed to identify endotypes of a disease. For example, gene expression data from a population of subjects with lupus may be used to identify a set of endotypes that differentiate patients within a population as having different subsets of the disease. In some cases, gene expression analysis may comprise analyzing gene expression changes of gene sets. For example, a plurality of gene sets may be analyzed and gene expression analysis of genes within the gene sets of the plurality of gene sets may be used to determine one or more endotypes. Gene expression changes of genes in a gene set, as described herein, may comprise increased gene expression of all genes in the gene set, increased gene expression of a subset of genes within the gene set, decreased gene expression of a subset of genes within a gene set, decreased gene expression of all genes in the gene set, or no change in expression in a subset of genes of the gene set. In some cases, a subset of genes of a gene set may have increased gene expression relative to one or more other references (e.g. reference samples), decreased expression relative to one or more other references, or a combination thereof. In some cases, a gene expression value may be determined for a gene set for a sample or a collection of samples. The gene expression value of the gene set may reflect a composite gene expression trend for the genes within the gene set or a subset of genes within the gene set. An endotype, as described herein, may be determined based on gene expression analysis of one or more gene sets as described herein. For example, a plurality of 10 gene sets may be used to determine one or more endotypes. Gene expression of genes within gene sets of the plurality of 10 gene sets may be determined and analyzed to identify one or more endotypes. The one or more endotypes may be used to classify a patient population, to determine a treatment regimen, to assess treatment efficacy, or any combination thereof. In some cases, one or more endotypes may be determined based on data from a plurality of subjects. For example, samples from a cohort of subjects with a disease and/or control subjects, may be analyzed to identify one or more endotypes that differentiate between subsets of subjects with the disease. In some cases, one or more samples of a subject may be analyzed to identify an endotype of the subject. The endotype of the subject may be identified based on a comparison to one or more endotypes identified based on a cohort of subjects with a disease. For example, samples of a cohort of subjects may be analyzed to identify a plurality of endotypes. A sample from another subject may be analyzed to identify an endotype of the other subject based on analysis of the subject's sample using similar gene sets used in determining the cohort endotypes.

The methods described herein may involve identifying one or more endotypes. The one or more endotypes may be based on analysis of expression data from a cohort of subjects, an individual subject, or a combination thereof. The one or more endotypes of the methods described herein may comprise at least 1 endotype, at least about 2 endotypes, at least about 3 endotypes, at least about 4 endotypes, at least about 5 endotypes, at least about 6 endotypes, at least about 7 endotypes, at least about 8 endotypes, at least about 9 endotypes, at least about 10 endotypes, at least about 11 endotypes, at least about 12 endotypes, at least about 13 endotypes, at least about 14 endotypes, at least about 15 endotypes, at least about 20 endotypes, at least about 25 endotypes, at least about 30 endotypes, at least about 35 endotypes, at least about 40 endotypes, at least about 50 endotypes, or more endotypes. The one or more endotypes of the methods described herein may comprise at most 1 endotype, at most about 2 endotypes, at most about 3 endotypes, at most about 4 endotypes, at most about 5 endotypes, at most about 6 endotypes, at most about 7 endotypes, at most about 8 endotypes, at most about 9 endotypes, at most about 10 endotypes, at most about 11 endotypes, at most about 12 endotypes, at most about 13 endotypes, at most about 14 endotypes, at most about 15 endotypes, at most about 20 endotypes, at most about 25 endotypes, at most about 30 endotypes, at most about 35 endotypes, at most about 40 endotypes, at most about 50 endotypes, or fewer endotypes. The one or more endotypes of the methods described herein may comprise about 1-50 endotypes, about 2-40 endotypes, about 3-35 endotypes, about 4-30 endotypes, about 5-25 endotypes, about 6-20 endotypes, about 7-15 endotypes, about 8-14 endotypes, about 9-13 endotypes, or about 10-12 endotypes.

The one or more endotypes of the methods described herein may be based on analysis of one or more gene sets, as described herein. In some cases, the one or more endotypes may be based on at least about 1 endotype, at least about 2 endotypes, at least about 3 endotypes, at least about 4 endotypes, at least about 5 endotypes, at least about 6 endotypes, at least about 7 endotypes, at least about 8 endotypes, at least about 9 endotypes, at least about 10 endotypes, at least about 11 endotypes, at least about 12 endotypes, at least about 13 endotypes, at least about 14 endotypes, at least about 15 endotypes, at least about 20 endotypes, at least about 25 endotypes, at least about 30 endotypes, at least about 35 endotypes, at least about 40 endotypes, at least about 50 endotypes, at least about 60 endotypes, at least about 70 endotypes, at least about 80 endotypes, at least about 90 endotypes, at least about 100 endotypes, at least about 125 endotypes, at least about 150 endotypes, at least about 175 endotypes, at least about 200 endotypes, or more endotypes. In some cases, the one or more endotypes may be based on at most about 1 endotype, at most about 2 endotypes, at most about 3 endotypes, at most about 4 endotypes, at most about 5 endotypes, at most about 6 endotypes, at most about 7 endotypes, at most about 8 endotypes, at most about 9 endotypes, at most about 10 endotypes, at most about 11 endotypes, at most about 12 endotypes, at most about 13 endotypes, at most about 14 endotypes, at most about 15 endotypes, at most about 20 endotypes, at most about 25 endotypes, at most about 30 endotypes, at most about 35 endotypes, at most about 40 endotypes, at most about 50 endotypes, at most about 60 endotypes, at most about 70 endotypes, at most about 80 endotypes, at most about 90 endotypes, at most about 100 endotypes, at most about 125 endotypes, at most about 150 endotypes, at most about 175 endotypes, at most about 200 endotypes, or fewer endotypes. In some cases, the one or more endotypes may be based on about 1-200 endotypes, about 2-175 endotypes, about 3-150 endotypes, about 4-125 endotypes, about 5-100 endotypes, about 6-90 endotypes, about 7-80 endotypes, about 8-70 endotypes, about 9-60 endotypes, about 10-50 endotypes, about 11-40 endotypes, about 12-35 endotypes, about 13-30 endotypes, about 14-25 endotypes, or about 15-20 endotypes.

The one or more endotypes as described herein may be based on gene expression of genes (e.g. of genes of one or more gene sets). Gene expression of genes may be determined based on a variety of ways. In some cases, RNA may be detected from one or more samples as described herein. For example, determining one or more endotypes may comprise performing RNA-sequencing (RNA-seq). RNA-seq may comprise generating a library preparation, sequencing the library preparation to generate sequence reads, aligning the sequence reads, determining a count for RNA of the input material, or any combination thereof. In some cases, RNA may be isolated and/or purified from a whole blood sample, as described herein The RNA may be reverse transcribed to generate cDNA. The cDNA may be processed to generate a library preparation. The library preparation may comprise nucleic acid sequences (e.g. DNA sequences). The nucleic acid sequences may comprise one or more molecular barcodes, one or more sample barcodes, one or more adaptors, or a combination thereof. The library preparation may be sequenced using any sequencing platform, including but not limited to an Illumina HiSeq 2500 sequencer, an Illumina X10 sequencer, an Illumina MiSeq sequencer, an Illumina NovaSeq sequencers, an Infinium EPIC Array, an Infinium FSA Array, an Infinium Omni Express sequencer, an Oxford Nanopore PromethION sequencer, a PacBio Sequel I sequencer, a PacBio Sequel II sequencer, an AVITI™ sequencer, an UG100™ sequencer, a DNBSEQ-E25 sequencer, a DNBSEQ-G99 sequencer, a DNBSEQ-G400 sequencer, a DNBSEQ-G800 sequencer, a DNBSEQ-T7 sequencer, a DNBSEQ-T20x2, a 454 sequencer, an IonTorrent sequencer, or a combination thereof.

Sequencing the library preparation may generate sequencing reads (e.g. a plurality of sequence reads). The sequencing reads may include information related to the nucleic acid sequence of the RNA from one or more samples, information about one or more barcodes (e.g. molecular barcodes and/or sample barcodes), or a combination thereof. The sequence reads may be listed in a variety of formats. For example, the sequence reads may be in one or more tables, one or more arrays, one or more files, one or more lists, or a combination thereof. The one or more files comprising sequence reads may comprise a variety of formats including but not limited to a BAM file, a CRAM file, a SFF file, a HDF5 file, a FASTQ file, a CSFASTA file, a QUAL file, a TXT file, or a combination there of.

The sequence reads described herein may comprise at least about 1,000, at least about 2,000, at least about 3,000, at least about 4,000, at least about 5,000, at least about 6,000, at least about 7,000, at least about 8,000, at least about 9,000, at least about 10,000, at least about 20,000, at least about 30,000, at least about 40,000, at least about 50,000, at least about 60,000, at least about 70,000, at least about 80,000, at least about 90,000, at least about 100,000, at least about 200,000, at least about 300,000, at least about 400,000, at least about 500,000, at least about 600,000, at least about 700,000, at least about 800,000, at least about 900,000, at least about 1,000,000, at least about 10,000,000, at least about 50,000,000, at least about 100,000,000, at least about 500,000,000, at least about 1,000,000,000, or more sequence reads. The plurality of sequence reads may comprise at most about 1,000, at most about 2,000, at most about 3,000, at most about 4,000, at most about 5,000, at most about 6,000, at most about 7,000, at most about 8,000, at most about 9,000, at most about 10,000, at most about 20,000, at most about 30,000, at most about 40,000, at most about 50,000, at most about 60,000, at most about 70,000, at most about 80,000, at most about 90,000, at most about 100,000, at most about 200,000, at most about 300,000, at most about 400,000, at most about 500,000, at most about 600,000, at most about 700,000, at most about 800,000, at most about 900,000, at most about 1,000,000, at most about 10,000,000, at most about 50,000,000, at most about 100,000,000, at most about 500,000,000, at most about 1,000,000,000, or less sequence reads. The plurality of sequence reads may comprise about 100,000 to about 1,000,000,000, about 200,000 to about 900,000,000, about 300,000 to about 80,0000,000, about 400,000 to about 70,0000,000, about 500000 to about 600,000,000, about 600,000 to about 500,000,000, about 700,000 to about 400,000,000, about 800,000 to about 300,000,000, about 900,000 to about 200,000,000, about 1,000,000 to about 100,000,000, or about 5,000,000 to about 50,000,000 sequence reads.

The plurality of sequence reads may be based on a plurality of input sequences (e.g. RNA molecules). The plurality of input sequences may comprise at least about 1,000, at least about 2,000, at least about 3,000, at least about 4,000, at least about 5,000, at least about 6,000, at least about 7,000, at least about 8,000, at least about 9,000, at least about 10,000, at least about 20,000, at least about 30,000, at least about 40,000, at least about 50,000, at least about 60,000, at least about 70,000, at least about 80,000, at least about 90,000, at least about 100,000, at least about 200,000, at least about 300,000, at least about 400,000, at least about 500,000, at least about 600,000, at least about 700,000, at least about 800,000, at least about 900,000, at least about 1,000,000, at least about 10,000,000, at least about 50,000,000, at least about 100,000,000, at least about 500,000,000, at least about 1,000,000,000, or more input sequences. The plurality of input sequences may comprise at most about 1,000, at most about 2,000, at most about 3,000, at most about 4,000, at most about 5,000, at most about 6,000, at most about 7,000, at most about 8,000, at most about 9,000, at most about 10,000, at most about 20,000, at most about 30,000, at most about 40,000, at most about 50,000, at most about 60,000, at most about 70,000, at most about 80,000, at most about 90,000, at most about 100,000, at most about 200,000, at most about 300,000, at most about 400,000, at most about 500,000, at most about 600,000, at most about 700,000, at most about 800,000, at most about 900,000, at most about 1,000,000, at most about 10,000,000, at most about 50,000,000, at most about 100,000,000, at most about 500,000,000, at most about 1,000,000,000, or less input sequences. The plurality of input sequences may comprise about 100,000 to about 1,000,000,000, about 200,000 to about 900,000,000, about 300,000 to about 80,0000,000, about 400,000 to about 70,0000,000, about 500000 to about 600,000,000, about 600,000 to about 500,000,000, about 700,000 to about 400,000,000, about 800,000 to about 300,000,000, about 900,000 to about 200,000,000, about 1,000,000 to about 100,000,000, or about 5,000,000 to about 50,000,000 input sequences.

The sequence reads, as described herein, may be aligned to one or more reference sequences Aligning the sequence reads to one or more reference sequences may be performed using a variety of algorithms and/or tools, including BLAST, STAR aligner, or a combination thereof. Aligning (e.g. mapping) the sequence reads may comprise comparing a sequence read of the plurality of sequence reads to one or more reference genomic sequences. The one or more reference sequences may comprise the NCBI Build 34 sequence, the NCBI Build 35 sequence, the NCBI Build 36.1 sequence, the GRCh37 sequence, the GRCh38 sequence, the T2T-CHM13 sequence, the GRCh39 sequence, the hg16 sequence, the hg17 sequence, the hg18 sequence, the hg19 sequence, the hs1 sequence, or a combination thereof. An alignment map may be generated based on aligning the sequence reads to one or more reference sequences. For example, a binary alignment map (BAM) may be generated. The alignment map may be used to generate read counts of genes of the sequence reads. The read counts of genes of the sequence reads may be used to determine gene expression levels and/or changes.

In some cases, gene expression analysis of genes may be performed based on a shared biological function. For example, a gene set comprising genes with a related biological function may be analyzed. In some cases, a gene set variation analysis (GSVA) algorithm may be performed. The GSVA algorithm may comprise a nonparametric, unsupervised analysis. In some cases, statistical analyses may be performed of gene expression data. The statistical analyses may be used to identify significant difference in gene expression amount gene sets. In some cases, one or more clustering methods may be performed. The one or more clustering methods may be used to identify one or more clusters. The one or more clusters may be used to identify one or more endotypes. For example, a k-means clustering analysis may be performed using gene expression data from samples collected form a cohort of subjects. The k-means clustering may identify an optimal number of clusters that differentiate between different subject subsets within the cohort based on gene expression differences. In another example, a random forest clustering analysis may be performed on gene expression data (e.g. gene expression data determined using a GSVA algorithm. In some cases, principal component analysis may be performed on the gene expression data. The one or more clustering methods may comprise hierarchical clustering, k-means clustering, self-organizing maps, model-based clustering, fuzzy clustering, density-based clustering, or any combination thereof. In some cases, hierarchical clustering may comprise Agglomerative nesting (AGNES), Unweighted Pair Group Method with Arithmetic Mean (UPGMA), Divisive Analysis (DIANA), Clustering Using Representatives (CURE), CHAMELEON, ROCK, Clustering Large Applications based upon RANdomized Search (CLARANS), Partitioning Around Medoids (PAM), Balanced Iterative Reducing and Clustering using Hierarchies (BIRCH), or any combination thereof. The self-organizing maps may comprise Chinese restaurant clustering (CRC), Chinese restaurant process (CRP), or any combination thereof. The fuzzy clustering may comprise Fuzzy C-Means (FCM) Clustering, Fuzzy K-means using Expectation Maximization (FKEM), Expectation Maximization (EM), or any combination thereof. In some cases, more than one clustering method may be used to identify a set of endotypes. In some cases, the set of endotypes identified by different clustering methods may be different (e.g. comprise different number of endotypes and/or different expression levels of genes of sets of genes). In some cases, the set of endotypes identified by different clustering methods may comprise the same number of endotypes. For example, in some cases two different clustering algorithms may be used to identify two different sets of endotypes. The two different sets of endotypes may comprise a different number of endotypes. The two different sets of endotypes may be used to identify whether a subject has a disease state, an optimal dose for a subject, the gradation of disease of a subject, or any combination thereof.

In some cases, the methods described herein for determining and/or identifying one or more endotypes may comprise using a computing system as described herein. In some cases, the computing system may be a cloud computing system. For example, the methods described herein may comprise using a cloud computing system to determine one or more endotypes (e.g. a first endotype).

### Computing System

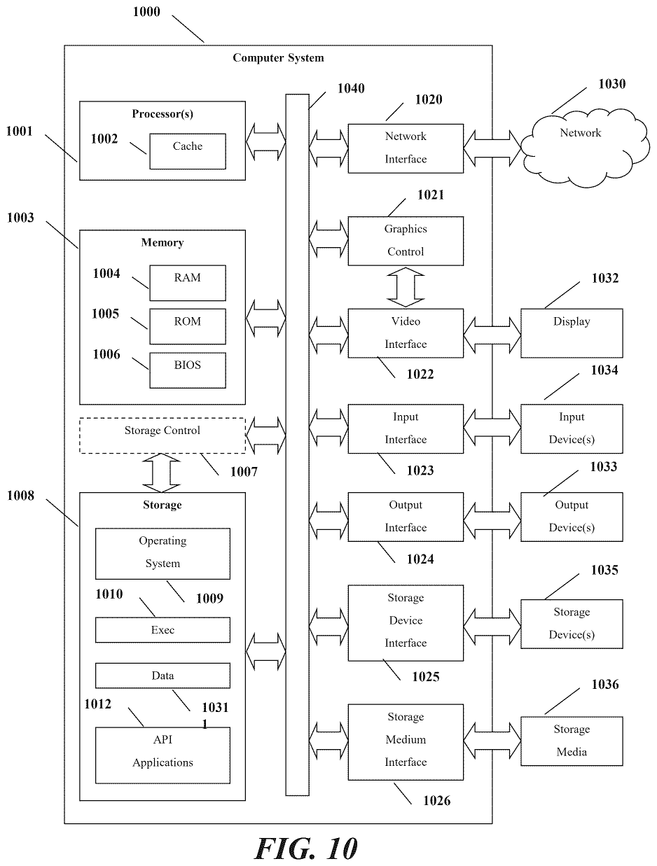

In some aspects, the present disclosure describes a computer-implemented system comprising: a digital processing device comprising: at least one processor, an operating system configured to perform executable instructions, a memory, and a computer program including instructions executable by the digital processing device to align sequence reads to a reference sequence, cluster expression data, or a combination thereof. In some aspects, the present disclosure describes a computer-implemented method, implementing any one of the methods disclosed herein in a computer system. Referring to FIG. 10, a block diagram is shown depicting an exemplary machine that includes a computer system 1000 (e.g., a processing or computing system) within which a set of instructions can execute for causing a device to perform or execute any one or more of the aspects and/or methodologies for determining one or more endotypes. The components in FIG. 10 are examples only and do not limit the scope of use or functionality of any hardware, software, embedded logic component, or a combination of two or more such components implementing particular embodiments.

Computer system 1000 may include one or more processors 1001, a memory 1003, and a storage 1008 that communicate with each other, and with other components, via a bus 1040. The bus 1040 may also link a display 1032, one or more input devices 1033 (which may, for example, include a keypad, a keyboard, a mouse, a stylus, etc.), one or more output devices 1034, one or more storage devices 10310, and various tangible storage media 1036. All of these elements may interface directly or via one or more interfaces or adaptors to the bus 1040. For instance, the various tangible storage media 1036 can interface with the bus 1040 via storage medium interface 1026. Computer system 1000 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1000 includes one or more processor(s) 1001 (e.g., central processing units (CPUs), general purpose graphics processing units (GPGPUs), or quantum processing units (QPUs)) that carry out functions. Computer system 1000 may be one of various high performance computing platforms. For instance, the one or more processor(s) 1001 may form a high-performance computing cluster. In some embodiments, the one or more processors 1001 may form a distributed computing system connected by wired and/or wireless networks. In some embodiments, arrays of CPUs, GPUs, QPUs, or any combination thereof may be operably linked to implement any one of the methods disclosed herein. Processor(s) 1001 optionally contains a cache memory unit 1002 for temporary local storage of instructions, data, or computer addresses. Processor(s) 1001 are configured to assist in execution of computer readable instructions. Computer system 1000 may provide functionality for the components depicted in FIG. 10 as a result of the processor(s) 1001 executing non-transitory, processor-executable instructions embodied in one or more tangible computer-readable storage media, such as memory 1003, storage 1008, storage devices 10310, and/or storage medium 1036. The computer-readable media may store software that implements particular embodiments, and processor(s) 1001 may execute the software. Memory 1003 may read the software from one or more other computer-readable media (such as mass storage device(s) 10310, 1036) or from one or more other sources through a suitable interface, such as network interface 1020. The software may cause processor(s) 1001 to carry out one or more processes or one or more steps of one or more processes described or illustrated herein. Carrying out such processes or steps may include defining data structures stored in memory 1003 and modifying the data structures as directed by the software.

The memory 1003 may include various components (e.g., machine readable media) including, but not limited to, a random access memory component (e.g., RAM 1004) (e.g., static RAM (SRAM), dynamic RAM (DRAM), ferroelectric random access memory (FRAM), phase-change random access memory (PRAM), etc.), a read-only memory component (e.g., ROM 10010), and any combinations thereof. ROM 10010 may act to communicate data and instructions unidirectionally to processor(s) 1001, and RAM 1004 may act to communicate data and instructions bidirectionally with processor(s) 1001. ROM 10010 and RAM 1004 may include any suitable tangible computer-readable media described below. In one example, a basic input/output system 1006 (BIOS), including basic routines that help to transfer information between elements within computer system 1000, such as during start-up, may be stored in the memory 1003.

Fixed storage 1008 is connected bidirectionally to processor(s) 1001, optionally through storage control unit 1007. Fixed storage 1008 provides additional data storage capacity and may also include any suitable tangible computer-readable media described herein. Storage 1008 may be used to store operating system 1009, executable(s) 1010, data 1011, applications 1012 (application programs), and the like. Storage 1008 can also include an optical disk drive, a solid-state memory device (e.g., flash-based systems), or a combination of any of the above. Information in storage 1008 may, in appropriate cases, be incorporated as virtual memory in memory 1003.

In one example, storage device(s) 10310 may be removably interfaced with computer system 1000 (e.g., via an external port connector (not shown)) via a storage device interface 10210. Particularly, storage device(s) 10310 and an associated machine-readable medium may provide non-volatile and/or volatile storage of machine-readable instructions, data structures, program modules, and/or other data for the computer system 1000. In one example, software may reside, completely or partially, within a machine-readable medium on storage device(s) 10310. In another example, software may reside, completely or partially, within processor(s) 1001.

Bus 1040 connects a wide variety of subsystems. Herein, reference to a bus may encompass one or more digital signal lines serving a common function, where appropriate. Bus 1040 may be any of several types of bus structures including, but not limited to, a memory bus, a memory controller, a peripheral bus, a local bus, and any combinations thereof, using any of a variety of bus architectures. As an example, and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1000 may also include an input device 1033. In one example, a user of computer system 1000 may enter commands and/or other information into computer system 1000 via input device(s) 1033. Examples of an input device(s) 1033 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1033 may be interfaced to bus 1040 via any of a variety of input interfaces 1023 (e.g., input interface 1023) including, but not limited to, serial, parallel, game port, USB, FIREWIRE, THUNDERBOLT, or any combination of the above. In some embodiments, an input device 1033 may be used to share data.

In particular embodiments, when computer system 1000 is connected to network 1030, computer system 1000 may communicate with other devices, specifically mobile devices and enterprise systems, distributed computing systems, cloud storage systems, cloud computing systems, and the like, connected to network 1030. Communications to and from computer system 1000 may be sent through network interface 1020. For example, network interface 1020 may receive incoming communications (such as requests or responses from other devices) in the form of one or more packets (such as Internet Protocol (IP) packets) from network 1030, and computer system 1000 may store the incoming communications in memory 1003 for processing. Computer system 1000 may similarly store outgoing communications (such as requests or responses to other devices) in the form of one or more packets in memory 1003 and communicated to network 1030 from network interface 1020. Processor(s) 1001 may access these communication packets stored in memory 1003 for processing.

Examples of the network interface 1020 include, but are not limited to, a network interface card, a modem, and any combination thereof. Examples of a network 1030 or network segment 1030 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1030, may employ a wired and/or a wireless mode of communication. In general, any network topology may be used.

Information and data can be displayed through a display 1032. Examples of a display 1032 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1032 can interface to the processor(s) 1001, memory 1003, and fixed storage 1008, as well as other devices, such as input device(s) 1033, via the bus 1040. The display 1032 is linked to the bus 1040 via a video interface 1022, and transport of data between the display 1032 and the bus 1040 can be controlled via the graphics control 1021. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1032, computer system 1000 may include one or more other peripheral output devices 1034 including, but not limited to, an audio speaker, a printer, a storage device, and any combinations thereof. Such peripheral output devices may be connected to the bus 1040 via an output interface 1024. Examples of an output interface 1024 include, but are not limited to, a serial port, a parallel connection, a USB port, a FIREWIRE port, a THUNDERBOLT port, and any combinations thereof.

In addition, or as an alternative, computer system 1000 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, notepad computers, set-top computers, media streaming devices, handheld computers, Internet appliances, mobile smartphones, and tablet computers.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® Ios®, Research In Motion® BlackBerry OS®, Google® Android®, Microsoft® Windows Phone® OS, Microsoft® Windows Mobile® OS, Linux®, and Palm® WebOS®.

In some embodiments, a computer system 1000 may be accessible through a user terminal to receive user commands. The user commands may include line commands, scripts, programs, etc., and various instructions executable by the computer system 1000. A computer system 1000 may receive instructions to analyze data or schedule a computing job for the computer system 1000 to carry out any instructions.

### Non-Transitory Computer Readable Storage Medium

In some aspects, the present disclosure describes a non-transitory computer-readable storage media encoded with a computer program including instructions executable by one or more processors to analyze data using any one of the methods disclosed herein. In some embodiments, a non-transitory computer-readable storage media may comprise sequence read data and/or expression data. In some embodiments, the platforms, systems, media, and methods disclosed herein include one or more non-transitory computer readable storage media encoded with a program including instructions executable by the operating system of an optionally networked computing device.

In further embodiments, a computer readable storage medium is a tangible component of a computing device. In still further embodiments, a computer readable storage medium is optionally removable from a computing device. In some embodiments, a computer readable storage medium includes, by way of non-limiting examples, flash memory devices, solid state memory, magnetic disk drives, magnetic tape drives, optical disk drives, distributed computing systems including cloud computing systems and services, and the like. In some embodiments, the program and instructions are permanently, substantially permanently, semi-permanently, or non-transitorily encoded on the media.

### Computer Program

In some aspects, the present disclosure describes a computer program product comprising a computer-readable medium having computer-executable code encoded therein, the computer-executable code adapted to be executed to implement any one of the methods disclosed herein. In some embodiments, the platforms, systems, media, and methods disclosed herein include at least one computer program, or use of the same.

A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages. In some embodiments, APIs may comprise various languages, for example, languages in various releases of TensorFlow, Theano, Keras, PyTorch, or any combination thereof which may be implemented in various releases of Python, Python3, C, C#, C++, MatLab, R, Java, or any combination thereof.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more standalone applications, one or more web browser plug-ins, extensions, add-ins, or add-ons, or combinations thereof.

### Web Application

In some embodiments, a computer program includes a web application. In some embodiments, a user may enter a query for analyzing RNA sequence data through a web application. In some embodiments, a user may upload data through a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (Ios) SDK, Android™ SDK, BlackBerry® SDK, BREW SDK, Palm® OS SDK, Symbian SDK, webOS SDK, and Windows® Mobile SDK.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of information about RNA sequence data, or any combination thereof. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Determination and Analysis of Endotypes for SLE Patients Undergoing Treatment with Iberdomide

Iberdomide is a cereblon-modulating E3 ubiquitin ligase that promotes proteasomal degradation of the hematopoietic transcription factors Ikaros and Aiolos. A phase 2 clinical trial of iberdomide demonstrated its efficacy for the treatment of subjects with systemic lupus erythematosus (SLE). A treatment response to iberdomide as measured by SLE Responder Index 4 (SRI-4) was observed with an effect size of 19% suggesting that heterogeneity in trial subjects impacted response to iberdomide therapy. We carried out analysis of RNA-seq from subjects in the iberdomide trial to identify molecular endotypes with distinct immune profiles at baseline and differences in clinical response over 24 weeks of treatment. K-means clustering of baseline gene expression profiles yielded five molecular endotypes (A-E) ranging from the least to most severe based on the degree of enrichment of inflammatory gene modules. Iberdomide induced shared pharmacodynamic effects on immune cell signatures across all endotypes that were essential for clinical response. Subjects in two endotypes (C and E) exhibited distinct molecular profiles that resulted in a significant response to treatment based on SRI-4, indicating that the molecular impact of iberdomide can vary based on endotype membership.

This study demonstrates that a molecular-based, unsupervised clustering approach to subset lupus subjects into distinct molecular endotypes may yield useful information regarding the abnormalities contributing to inflammation and immune system dysfunction at an individual patient level. Identification of subjects with molecular profiles indicative of clinical responsiveness offers the possibility of enriching trials for responsive subjects and selecting patients for specific therapies in clinical care. Determining molecular biomarkers indicative of pharmacodynamic and subset-specific drug effects necessary for clinical response provides the possibility of monitoring the molecular impact of a drug to guide effective therapy.

Systemic lupus erythematosus (SLE) is a chronic autoimmune disease with a wide array of clinical features, including unexpected disease flares and risk of irreversible organ damage (1,2). Because of its heterogeneity, it is difficult to predict the likelihood of flare or response to treatment in an individual patient. (3-7). We recently reported that lupus subjects can be divided into definable subsets based on their gene expression profile, and that membership in individual subsets has clinical implications, including likelihood of severe flare and responsiveness to medication (8). The subsets manifested specific gene expression profiles but only modestly different clinical and standard laboratory abnormalities, suggesting that they represented disease endotypes or groups of subjects with distinct immunopathogenic features.

Recent reports have shown that iberdomide can be effective in treating subjects with SLE (9-11). Iberdomide acts as a high-affinity ligand for the intracellular protein cereblon that, after engagement, promotes ubiquitylation and proteasomal degradation of the hematopoietic transcription factors Ikaros and Aiolos (12,13). Results from a proof-of-concept phase 2a study of iberdomide in which clinical and biomarker information was collected from subjects with SLE showed that administration of the agent decreased B cells and plasmacytoid dendritic cells (pDCs), along with inhibiting autoantibody production and type I interferon (IFN) production (9). More recently, a larger phase 2b global, randomized, placebo-controlled trial evaluating the efficacy and safety of iberdomide in 288 subjects with active SLE (ClinicalTrials.gov NCT03161483) showed that more than half (54%) of the 81 subjects receiving the highest dose of iberdomide (0.45 mg once daily) achieved a significant treatment response based on the SLE Responder Index 4 (SRI-4) (14) at week 24, compared to 35% of the 83 subjects receiving placebo (10). Whole blood gene expression data was also obtained from a subset of participants in the phase 2b study. Effectiveness was particularly noteworthy in subjects with an elevated IFN gene signature (IGS) (11). These findings suggested that subsets of SLE subjects might be especially responsive to iberdomide.

In the present study, we employed molecular subsetting of the iberdomide trial study subjects to determine their baseline molecular endotypes. The study involved 1) determining the gene expression-based endotypes of the subjects who were responsive to iberdomide treatment; and 2) analyzing the impact of this immunomodulatory agent on gene expression profiles of SLE subjects over the 24 weeks of the trial.

### Methods

Samples. Whole blood samples were obtained from a subset of participants in the phase 2b clinical trial of iberdomide in subjects with SLE (ClinicalTrials.gov NCT03161483) (10). Notably, all subjects in the present study sample were female. All subjects were randomly assigned to receive placebo or one of three different doses of iberdomide (0.15, 0.3 or 0.45 mg/day) administered once daily for 24 weeks.

RNA-Seq analysis. Paired-end FASTQ files of 50M base pairs were generated from both the baseline (pretreatment day 1) and longitudinal (week 4, 12 and 24) blood samples. The raw RNA-Seq data were processed through a high-throughput sequencing pipeline using a series of command line tools for high-performance computing within the Google Cloud Platform. For initial quality control, the raw data (FASTQ) files were processed using FASTQC, a standalone tool resulting in a FASTQC report that identifies bad quality reads (if any) and eliminates adapter contamination. Thereafter, the paired-end FASTQ files were further processed using Trimmomatic (version 0.38), which is a next-generation sequencing tool that removes bad-quality reads and adapters using ilmnclip, headcrop and sliding window filters. The processed reads were mapped to the human reference genome hg38 using STAR aligner. This generated sequence alignment map (SAM) files, followed by conversion to comprehensive binary alignment map (BAM) files using SAMBAMBA. Read counts were summarized using the featureCounts function in the Subread package.

Gene set variation analysis (GSVA). The GSVA algorithm can utilize a nonparametric, unsupervised approach to enable pathway-centric analyses of RNA-Seq data, in which gene-sample matrices may be transformed into gene-set-sample matrices. GSVA can calculate sample-wise gene enrichment scores using predefined gene sets. Instead of assessing gene expression changes on a population level, GSVA focuses on the individual sample relative to the rest of the samples in a group. IQR filtration for removal of outliers (IQR>0) was applied to remove low intensity genes. GSVA enrichment scores were calculated using a nonparametric approach that employs a Kolmogorov-Smirnoff-like random walk statistic, with resultant scores ranging from −1 (low enrichment) to +1 (high enrichment). These enrichment scores represent the largest negative and positive random walk deviation from zero, for both the individual sample and the gene set.

Statistical analysis. Paired t-test and repeated measures analysis of variance were used to calculate the significance of differences in gene expression among the gene sets identified in paired lupus blood samples across four treatment groups: 1) placebo, 2) 0.15 mg iberdomide, 3) 0.3 mg iberdomide and 4) 0.45 mg iberdomide. These analyses were performed at week 4, week 12 and week 24, as compared to baseline. The level of significance was determined using a Bonferroni-corrected P value threshold of less than or equal to 0.05. Differences in GSVA scores between groups and their directionality were assessed using Hedges' g along with the Cohen's d function as a measure of effect size (Bioconductor in R package). Welch's t-test, with Bonferroni correction, was used to assess statistically significant differences in baseline clinical characteristics between subjects in each of the gene expression-based molecular endotypes. The threshold of significance was set at a P value less than or equal to 0.05. Effect sizes among the groups were visualized as heatmaps (ComplexHeatmap, Bioconductor in R package). GSVA scores were visualized as violin plots using the violin plots function in ggplot2 (Bioconductor in R package).

K-means clustering, an algorithm for unsupervised analysis of gene expression data, was applied to group the baseline lupus blood samples into molecular endotypes based on the GSVA scores of varying molecular features. To determine the optimal number of clusters, we utilized KElbowVisualizer in Python. This tool implements the elbow method and uses the silhouette score as a scoring parameter. By fitting the model with a range of values for “K,” we identified the most appropriate number of clusters for the data.

We computed a gene-based composite score of immunologic activity, the Lupus Severity Composite Score (LSCS), for each lupus sample. To calculate the LSCS score, GSVA enrichment scores from all 32 modules of molecular features in each lupus blood sample were binarized into 0 (negative GSVA score) or 1 (positive GSVA score). A ridge-penalized logistic regression model with input of GSVA enrichment scores from individual samples was employed to obtain regression coefficients, and these coefficients were multiplied by the binarized GSVA scores, as described previously (4). The product scores for each sample were then summed to create the LSCS. The summed LSCS scores were visualized as violin plots using the violin plots function in ggplot2 (Bioconductor in R package).

Associations between the LSCS of each lupus blood sample at baseline and each patient's clinical characteristics at baseline, including SLEDAI-2K scores, anti-double-stranded DNA (anti-dsDNA) status, and serum levels of complement components C3 and C4, were examined using Pearson's correlation analysis. Correlations were visualized using the scatter plots function in ggplot2 (Bioconductor in R package).

Interpretation of model predictions using Shapley Additive exPlanations (SHAP). SHAP analysis was used to identify the most important features that characterized the lupus blood samples from week 24 versus baseline within clinical responder and nonresponder groups of subjects. The waterfall plot technique from the SHAP library in Python was used to interpret the model's prediction at the individual sample level. The waterfall plots depict the positive/negative contribution of each feature toward the model prediction calculated by f(x) within each sample. A positive contribution of any feature may imply that the feature value may be responsible for increasing its model predictive performance, whereas a negative value means the feature value led to a decrease in its model prediction. To understand the contribution of each feature toward model output in greater detail, a binary classification model using a random forest (RF) classifier was built using data from baseline and week 24 samples to classify individual subjects as either SRI-4 clinical responders or nonresponders at week 24 following treatment with the highest dose of iberdomide (0.45 mg). This RF model incorporated the same 32 curated gene modules as features. The RF model-built data were then analyzed using SHAP to identify key features contributing to the final model predicted outcome. The positive and negative contribution scores of each feature were visualized as heatmaps (ComplexHeatmap, Bioconductor in R package).

Principal Component Analysis (PCA). Dimensionality reduction techniques, such as Principal Component Analysis (PCA), were applied to the relevant clinical features (age, SLEDAI, BILAG, C3, C4, antidsDNA, CLASIA, and CLASID) and categorical features (ancestry, RNP, SSA, SSB, and anti-SM) from baseline data to cluster the data points according to the SRI4 response. Before the analysis, the data were normalized using the StandardScaler from the sklearn.preprocessing module, followed by PCA on the standardized data. Two principal components were extracted to reduce the data to two dimensions and capture the variance within the dataset. The explained variance ratios for the first and second components were calculated to quantify the proportion of the total variance accounted for by each component. A scatter plot, generated using the Seaborn library, was used to visualize the reduced-dimensional data. The x- and y-axes corresponded to the first and second principal components (“PCA1” and “PCA2”), respectively. Data points were color-coded based on the SRI4 response labels: blue for “Non-responders,” red for “Responders”, and gray for “NA.”

### Results

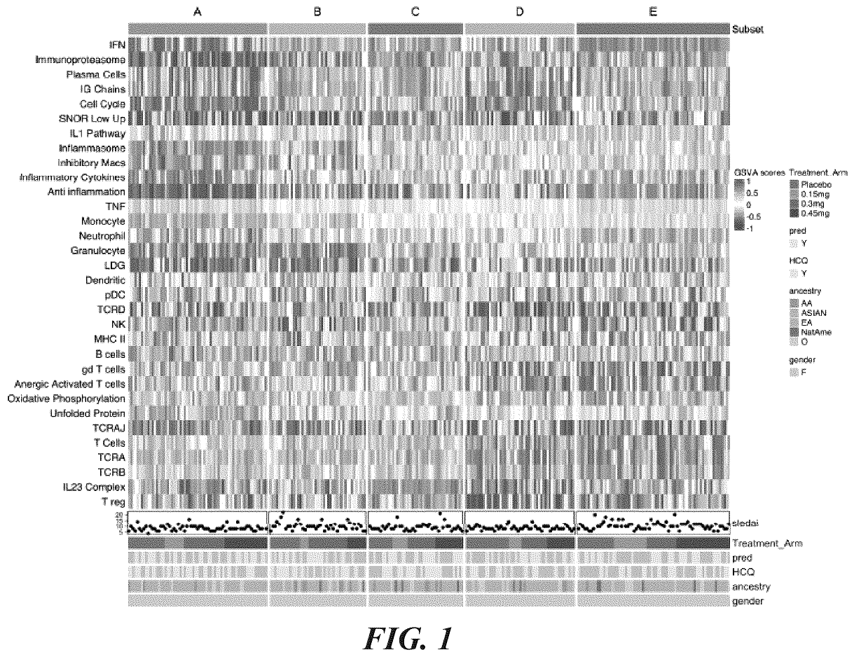

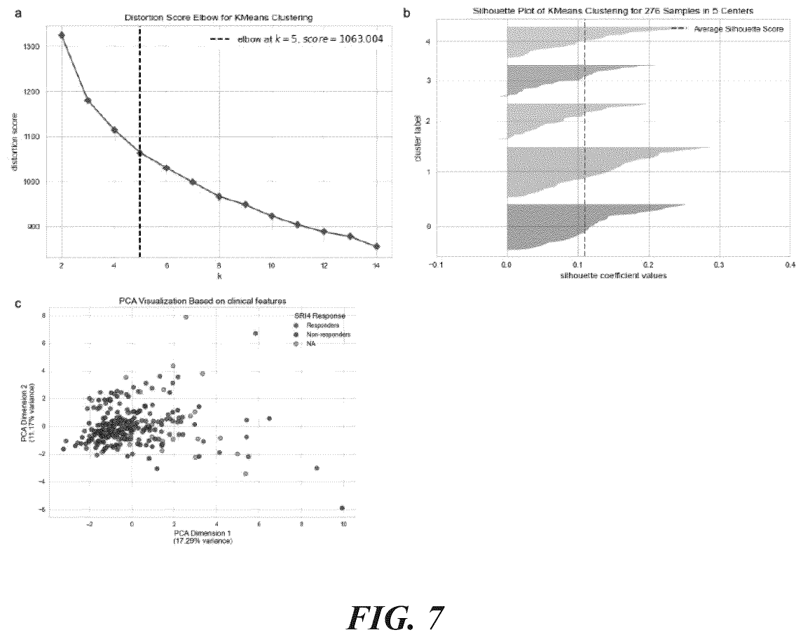

Molecular clusters from baseline blood samples. Initially, the number of molecular subsets was identified using comprehensive K-means clustering of GSVA scores for the 32 cellular and process gene modules in baseline blood samples from 276 SLE subjects. Elbow and silhouette plots were analyzed to determine the optimal number of subsets (K values) (FIG. 7), and five distinct molecular endotypes were identified, designated endotypes A-E (FIG. 1).

Among the five molecular endotypes, endotype A was characterized by significantly decreased enrichment (GSVA score<0) of gene modules related to the IFN gene signature (IGS), as well as the tumor necrosis factor (TNF), interleukin-1 (IL-1), anti-inflammation, plasma cell and Ig chain signatures, and increased enrichment (GSVA score>0) of modules defining B cells, regulatory T (Treg) cells, T cells and T cell receptor modules (TCRA, TCRB, TCRD and TCRAJ) as compared to the other endotypes; this was defined as the “least abnormal” endotype. In contrast to endotype A, lupus endotype E was characterized by the highest enrichment of inflammatory gene modules, and was defined as the “most abnormal” endotype. The other three endotypes (endotypes B-D) displayed varying, intermediate patterns of immunologic activity. Notably, lupus endotypes B and C demonstrated predominantly increased expression of plasma cells and Ig chain gene modules. Enrichment of the IGS was observed in endotypes B, C and E and to a lesser extent in endotype D.

The numbers of subjects were evenly distributed among the five molecular endotypes, with the highest number of subjects observed in endotypes A and E (Table 1). The majority of subjects in all five endotypes were White subjects, whereas those of African ancestry were mostly in endotypes B and C, with gene expression profiles characterized by enrichment of the plasma cell and Ig gene signatures (17). Almost 50% of subjects in each lupus endotype had been treated with prednisone or hydroxychloroquine, although these frequencies were not significantly different (Table 1).

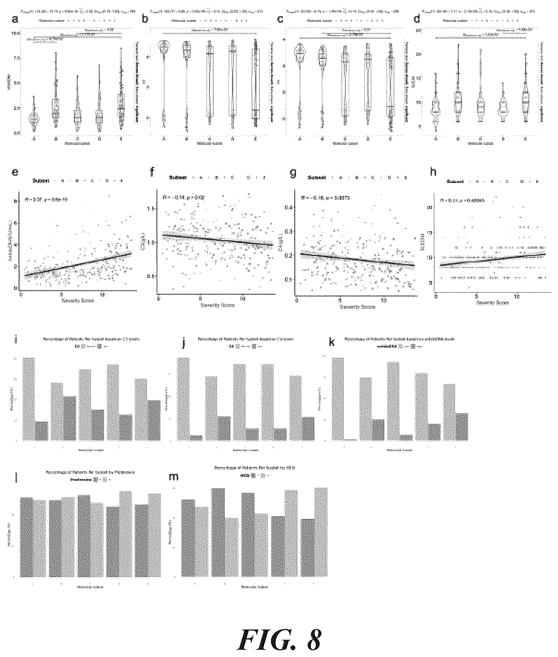

We compared the baseline clinical characteristics of the subjects according to each molecular endotype (Table 1 and FIG. 8). Subjects with endotype A exhibited significantly lower titers of anti-DNA compared to endotypes B and E. Moreover, significant differences in anti-dsDNA levels were also observed between endotypes C and E (FIG. 8). Similarly, complement C3 and C4 levels were significantly lower in endotype E compared to the other endotypes (FIG. 8). The frequency of anti-RNP antibodies was significantly higher in endotype E, but subjects with endotypes B and C also had higher anti-RNP levels (Table 1), the latter two endotypes being enriched in subjects of African ancestry (17-18). SLEDAI-2K scores of disease activity were modestly, but significantly higher in endotype E compared to the other endotypes (FIG. 8).

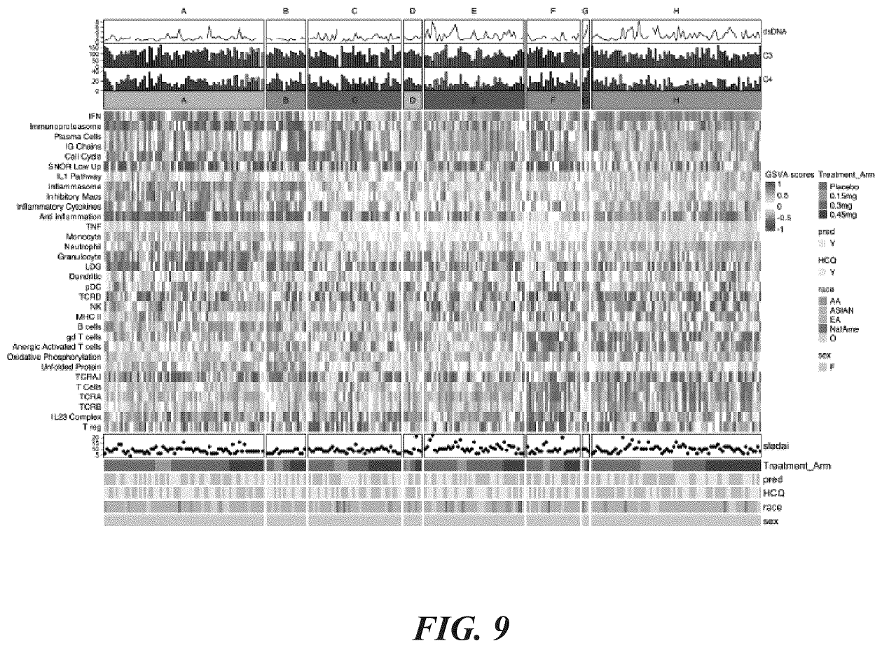

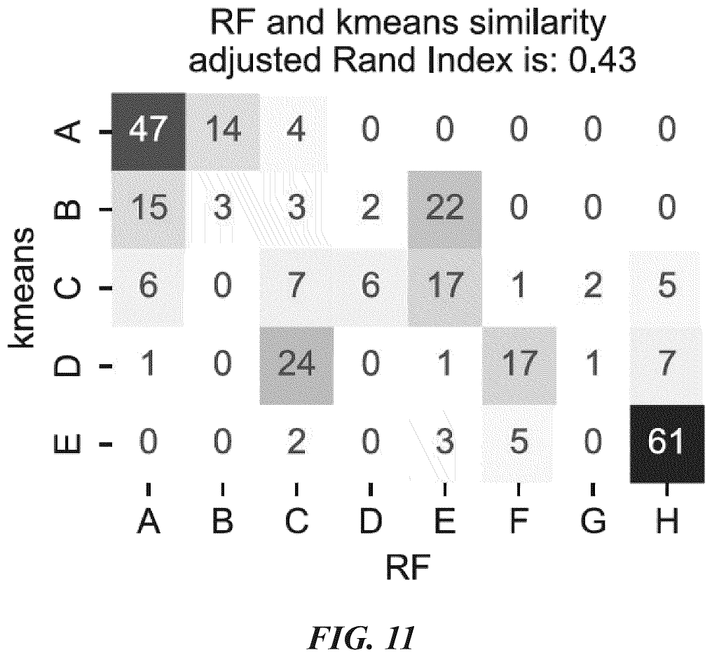

In order to relate the endotypes identified in this study to those reported previously in a study using 3,166 lupus subjects from 17 datasets, we applied our previously developed and validated machine learning (ML) algorithm (8). Using this orthogonal approach, we identified the same eight distinct lupus endotypes (denoted endotypes A-H) previously found (FIG. 9). For comparative analysis of these two methods, the molecular endotypes derived from each were compared with concordance assessed using the Adjusted Rand Index (ARI). Even though the two clustering approaches yielded different numbers of subsets (5 versus 8), the overall similarity of subsets was moderate (ARI-0.43). Notably, however, subjects classified as subset A, the least abnormal endotype by either method, and subjects classified as subsets E or H, the most abnormal endotypes by either method, had substantially similar clinical characteristics and gene expression patterns, with strong alignment of sample classifications, as indicated by the ARI of congruence (FIG. 11). Since the numbers of subjects classified in three of the subsets using the ML model (endotypes B, D and G) were quite small, the present analysis focused on the five molecular endotypes derived from K-means clustering.

TABLE 2
Baseline demographic and clinical characteristics of SLE
patients in the phase 2b iberdomide trial by endotype*

| | | Endotype | | | | | | | | | | |
|---|---|---|---|---|---|---|---|---|---|---|---|---|
| | Total | A | | B | | C | | D | | E | | |
| | assessed | (n = 65) | | (n = 45) | | (n = 44) | | (n = 51) | | (n = 71) | | P† |
| Age, mean ± SD years | 276 | 46.22 ± | | 43.51 ± | | 47.64 ± | | 49.00 ± | | 40.39 ± | | <0.001 |
| | | 11.05 | | 12.90 | | 12.92 | | 13.22 | | 12.19 | | |
| Sex, female, no. (%) | 276 | 55 | (100) | 35 | (100) | 27 | (100) | 45 | (100) | 55 | (100) | — |
| Race or ethnicity, no. | 276 | | | | | | | | | | | 0.4 |
| (%) | | | | | | | | | | | | |
| Native American or | | 0 | (0) | 2 | (4.4) | 3 | (6.8) | 2 | (3.9) | 5 | (7.0) | |
| Alaska Native | | | | | | | | | | | | |
| Asian | | 0 | (0) | 0 | (0) | 0 | (0) | 0 | (0) | 1 | (1.4) | |
| African American | | 3 | (4.6) | 7 | (16) | 5 | (11) | 3 | (5.9) | 1 | (1.4) | |
| White | | 52 | (80) | 26 | (58) | 33 | (75) | 37 | (73) | 55 | (77) | |
| Other | | 10 | (15) | 10 | (22) | 3 | (6.8) | 9 | (18) | 9 | (13) | |
| Treatment arm, no. (%) | 276 | | | | | | | | | | | 0.3 |
| Iberdomide | | | | | | | | | | | | |
| 0.15 mg | | 9 | (14) | 4 | (8.9) | 7 | (16) | 6 | (12) | 15 | (21) | |
| 0.3 mg | | 19 | (29) | 18 | (40) | 13 | (30) | 12 | (24) | 14 | (20) | |
| 0.45 mg | | 20 | (31) | 9 | (20) | 13 | (30) | 13 | (25) | 25 | (35) | |
| Placebo | | 17 | (26) | 14 | (31) | 11 | (25) | 20 | (39) | 17 | (24) | |
| SLEDAI-2K, mean ± | 275 | 8.56 ± | | 10.36 ± | | 9.34 ± | | 8.76 ± | | 10.68 ± | | <0.001 |
| SD | | 2.27 | | 3.37 | | 3.04 | | 1.87 | | 3.11 | | |
| BILAG, mean ± SD | 274 | 14.84 ± | | 17.16 ± | | 16.52 ± | | 15.66 ± | | 17.27 ± | | 0.14 |
| | | 6.07 | | 6.23 | | 4.62 | | 6.08 | | 6.22 | | |
| Anti-dsDNA, mean ± | 264 | 5.10 ± | | 137.57 ± | | 16.60 ± | | 37.22 ± | | 132.69 ± | | <0.001 |
| SD IU/ml | | 5.28 | | 501.95 | | 47.90 | | 137.62 | | 590.38 | | |
| C3, mean ± SD mg/dl | 273 | 90.24 ± | | 78.95 ± | | 59.61 ± | | 66.68 ± | | 46.77 ± | | <0.001 |
| | | 52.44 | | 47.01 | | 56.08 | | 57.81 | | 51.83 | | |
| C4, mean ± SD mg/dl | 268 | 16.78 ± | | 13.47 ± | | 10.51 ± | | 12.20 ± | | 7.79 ± | | <0.001 |
| | | 10.86 | | 9.18 | | 10.60 | | 12.04 | | 9.60 | | |
| Anti-RNP, no. (%) | 276 | | | | | | | | | | | 0.008 |
| Equivocal | | 0 | (0) | 1 | (2.2) | 0 | (0) | 0 | (0) | 0 | (0) | |
| Not done | | 2 | (3.1) | 1 | (2.2) | 2 | (4.5) | 0 | (0) | 1 | (1.4) | |
| Negative | | 48 | (74) | 21 | (47) | 24 | (55) | 43 | (84) | 41 | (58) | |
| Positive | | 15 | (23) | 22 | (49) | 18 | (41) | 8 | (16) | 29 | (41) | |
| Prednisone, no. (%) | 276 | | | | | | | | | | | >0.9 |
| Yes | | 33 | (51) | 22 | (49) | 23 | (52) | 23 | (45) | 33 | (46) | |
| No | | 32 | (49) | 23 | (51) | 21 | (48) | 28 | (55) | 38 | (54) | |
| Hydroxychloroquine, | 276 | | | | | | | | | | | 0.12 |
| no. (%) | | | | | | | | | | | | |
| Yes | | 34 | (52) | 27 | (60) | 25 | (57) | 21 | (41) | 28 | (39) | |
| No | | 31 | (48) | 18 | (40) | 19 | (43) | 30 | (59) | 43 | (61) | |

*SLEDAI-2K = Systemic Lupus Erythematosus (SLE) Disease Activity Index 2000; BILAG = British Isles Lupus Assessment Group disease activity index; anti-dsDNA = anti-double-stranded DNA.
†By Kruskal-Wallis rank sum test or Pearson's chi-square test.

Immune severity score calculation and clinical correlations. GSVA scores of the 32 gene modules from the baseline blood samples were reduced to a single composite score of immunologic activity, the Lupus Severity Composite Score (LSCS), to quantify the levels of molecular abnormalities in lupus subjects (8). The LSCS of each patient was correlated with various clinical variables to determine whether there was a relationship between the observed molecular abnormalities and clinical characteristics. The analyses showed that LSCS scores were positively correlated with anti-dsDNA levels (P=8.6×10⁻¹⁰) and SLEDAI-2K scores (P=0.00043) but negatively correlated with serum levels of C3 and C4 (P=0.02 and P=0.0073, respectively) (FIG. 8). Despite the significant but weak correlations observed, the findings suggest that the LSCS could capture clinically useful information.

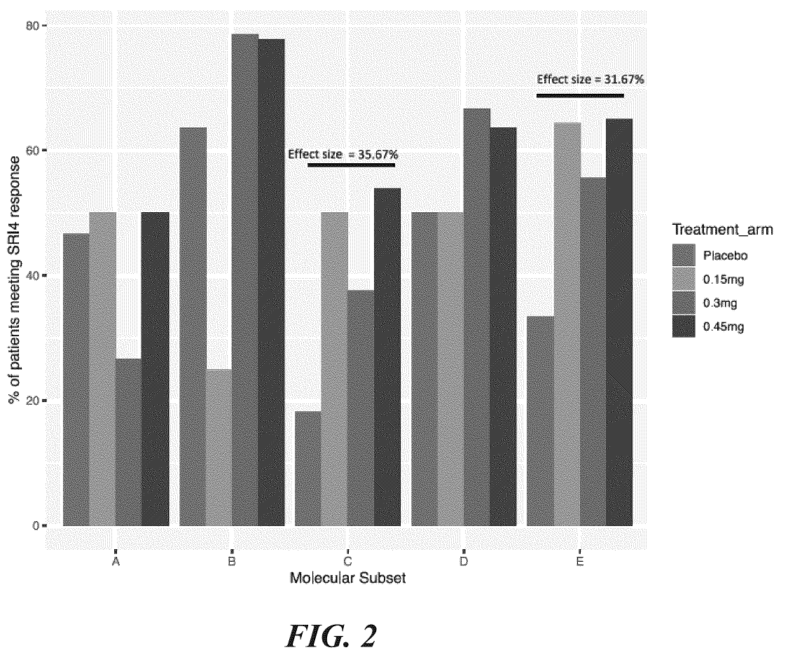

Molecular endotyping predicts therapeutic response to iberdomide. Treatment response was assessed using the SRI-4 response frequency (10) at week 24, the primary end point used in the trial (6). Analysis of the SRI-4 response revealed two molecular endotypes that showed a significant clinical response following 24 weeks of treatment with iberdomide, especially among subjects receiving the highest dose of iberdomide (0.45 mg once daily) although clinical response to the lowest dose (0.3 mg) was comparable to the highest dose in certain endotypes (FIG. 2). Specifically, among the endotypes, subjects with endotypes C and E showed the highest frequency of SRI-4 responses to iberdomide treatment (across all three dose groups) compared to placebo, with respective effect sizes of 35.67% and 31.67% (each P=0.002, by Fisher's exact test). As previously reported (ref), one of these endotypes (E) had the highest level of enrichment of the IGS, although the other responsive subset (C) had increased but not maximal enrichment of the IGS. Furthermore, enrichment of the IGS alone did not predict responsiveness to iberdomide, since one of the subsets in which an increase in IFN gene expression was observed (endotype B) did not show a clinical response. It is notable that, compared to endotypes C and E, the other three endotypes had higher placebo SRI-4 response rates and there was no difference in response rates between the active drug and placebo groups within these endotypes. These findings indicate that molecular endotyping may be useful in identifying subsets of SLE subjects who respond to iberdomide.

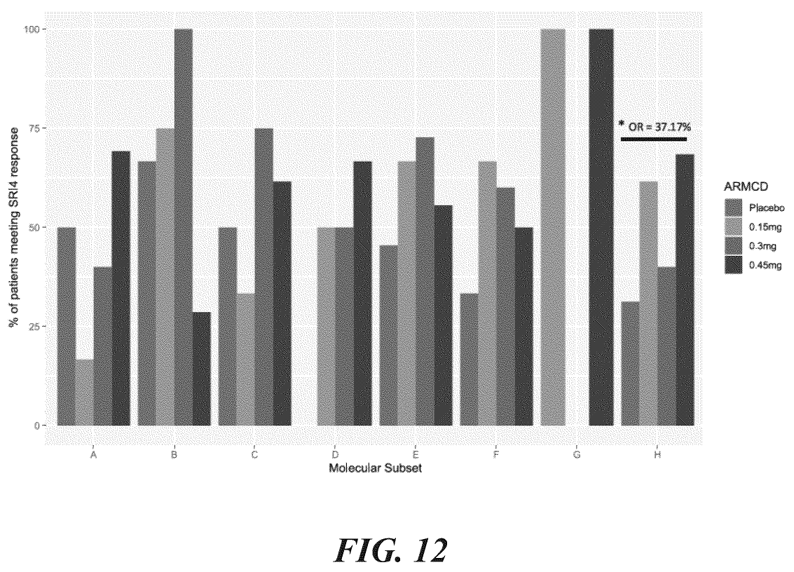

SRI-4 clinical response rates were also assessed in SLE subjects according to the RF model classification. Subjects within endotype H who were administered the highest dose of iberdomide (0.45 mg once daily) exhibited a significant SRI-4 clinical response, with an effect size of 37.17% (P=0.028 versus placebo at week 24, by Fisher's exact test, FIG. 12).

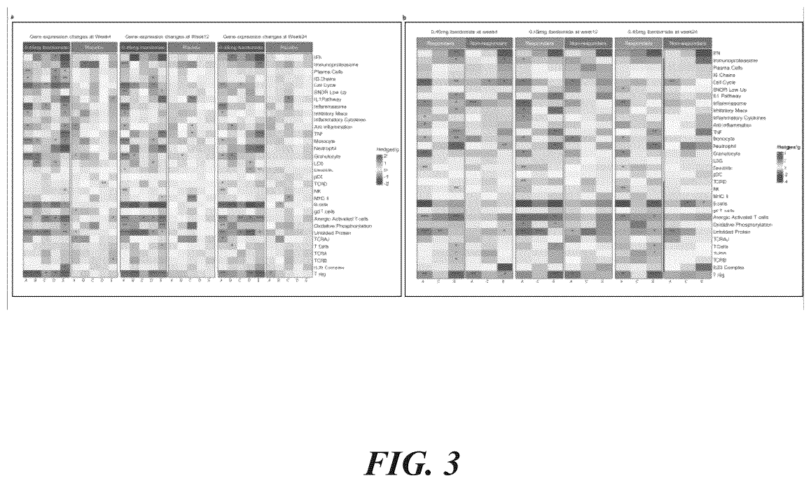

Association of longitudinal gene expression changes with treatment in different endotypes. Longitudinal gene expression analysis was conducted to evaluate the impact of iberdomide treatment across the various endotypes. This analysis identified gene expression changes associated with iberdomide treatment (at the highest dose) that were either common to all five endotypes or unique to each endotype (FIG. 3, left panel). Across all endotypes (A-E), iberdomide treatment consistently led to increased expression of anergic/activated T cells, Tregs and oxidative phosphorylation and decreased B cell signatures. Importantly, no significant gene expression changes across all endotypes were observed in subjects receiving placebo. These common changes likely reflect the pharmacodynamic effects of iberdomide. On the other hand, unique changes in gene expression among endotype E subjects were observed. Treatment with the highest dose of iberdomide resulted in significant changes by week 4, including reduced expression of the IGS, as well as the immunoproteasome, IL-1 pathway, inflammasome, inhibitory macrophage, anti-inflammatory, TNF, monocyte, neutrophil and IL-23 complex signatures. Many of these changes persisted through week 24, with significant decreases in the IGS, immunoproteasome, TNF and neutrophil signatures, and increased DC and unfolded protein response signatures. Most changes, except for the immunoproteasome signature, were also present at week 12. In contrast, subjects with endotype C exhibited fewer changes in gene expression profiles, despite a significant clinical response to the highest dose of iberdomide. Only an increase in cell cycle and unfolded protein signatures at both week 4 and week 24 was observed, in addition to the pharmacodynamic effects noted in all endotypes.

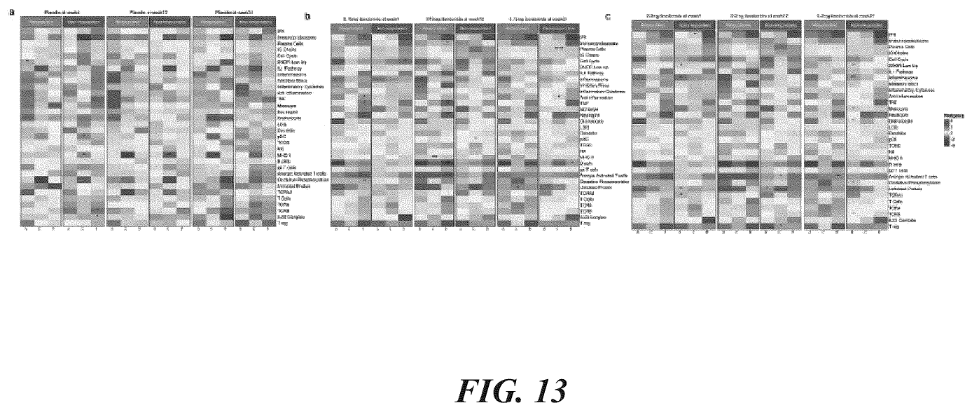

Treatment-induced changes in gene expression profiles among clinically responsive subjects. To relate changes in gene expression profiles to clinical responsiveness, we assessed transcriptional changes in clinical responders and nonresponders in the various endotypes (FIG. 13). Endotypes B and D were excluded because of insufficient number of samples for statistical analysis. Following treatment with iberdomide (0.45 mg), both the putative “pharmacodynamic” effects noted in endotype A and the pharmacodynamic and therapeutic effects noted in endotype E were largely confined to clinical responders (FIG. 3, right panel). Other than a significant increase in the unfolded protein response signature at weeks 4 and 12 in clinical responders, consistent changes in endotype C were not observed. Transcriptional changes were less prominent with lower doses of iberdomide (FIG. 13). Of note, no significant transcriptional changes were observed in clinical responders treated with placebo (FIG. 13).

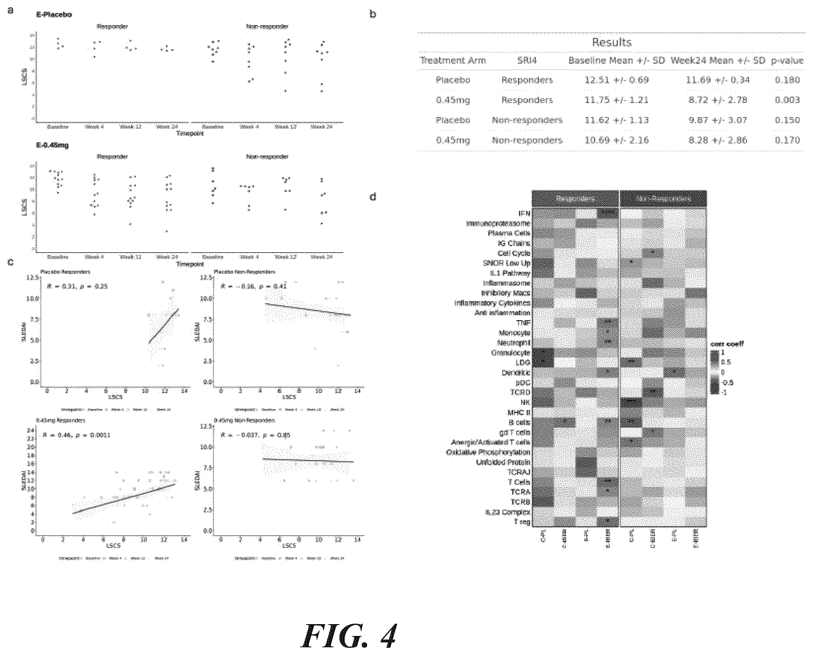

The effect of iberdomide on LSCS scores was also evaluated in endotype E, where responders showed a significant (p=0.003) change at week 24 compared to baseline (FIG. 4, top panel). Nonresponders did not exhibit significant changes, and no variations were noted in the placebo group. It is notable that there was a significant correlation between LSCS and clinical activity measured by SLEDAI only in responders to iberdomide, but not in nonresponders and not in placebo treated subjects (FIG. 4, bottom left (c)). To identify changes in specific molecular pathways that associated with clinical improvement, we correlated GSVA scores of each molecular module with SLEDAI. In endotype E responders, there was a significant positive correlation between the IGS, as well as the TNF, monocyte, neutrophil and B cell modules and a significant negative correlation between T cell and Treg modules (FIG. 4, bottom right (d)). In contrast, in endotype C responders, there was a positive correlation only with the B cell module. No correlations were noted in placebo treated responders. In nonresponders, only scattered associations were noted, and usually in different modules.

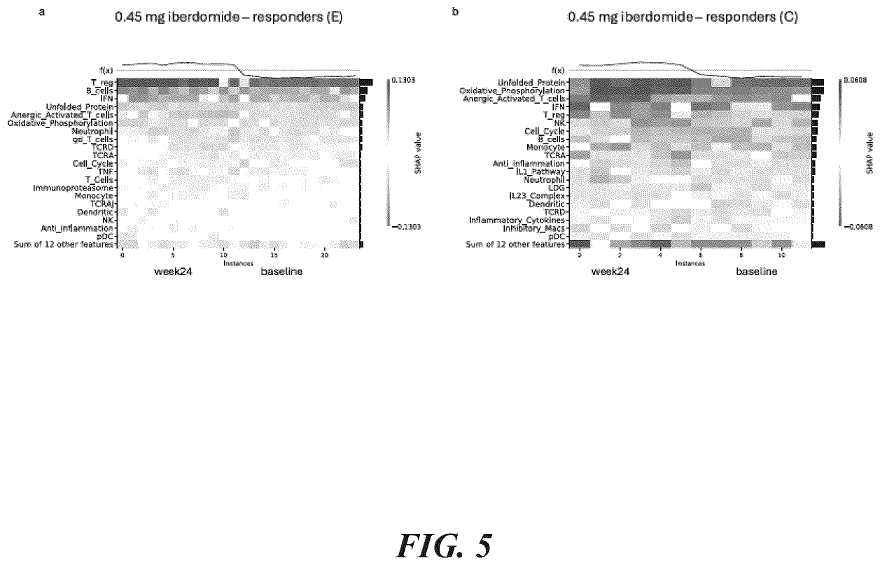

Feature importance analysis further delineates molecular determinants of clinical response. To assess the changes in gene expression profiles that most associated with clinical responsiveness in greater detail, we applied SHAP analysis, a method that identifies features that disproportionately affect classes of subjects, in this case clinical responders. For this analysis, we identified key features contributing to the classification of patients at 24 weeks compared to baseline in clinical responders following treatment with the highest dose of iberdomide (0.45 mg). These analyses were limited to subjects with endotypes C and E. Results of these analyses showed that Treg and B cell signatures and the IGS were the top contributors among endotype E responders (FIG. 5, left panel). Significant positive contributions from each of these variables toward the final model outcome were seen in 11 of 12 individual lupus blood samples (FIG. 5, left panel). In endotype C responders, the most important features included unfolded protein, oxidative phosphorylation and anergic/activated T cell signatures (FIG. 5, right panel).

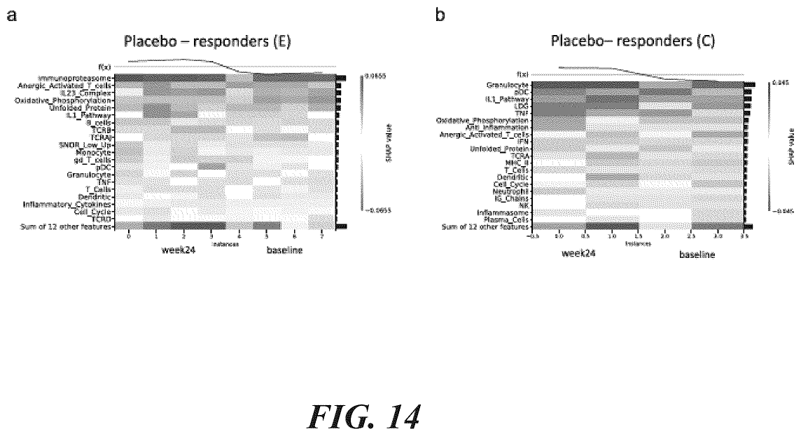

We also applied SHAP analysis to identify key features contributing to the clinical response in the placebo treated subjects (FIG. 14). In endotype E placebo responders, key contributors included the immunoproteasome, anergic/activated T cell and IL-23 complex signatures (FIG. 14). For endotype C placebo responders, significant features included granulocyte, pDC and the IL-1 signatures (FIG. 14).

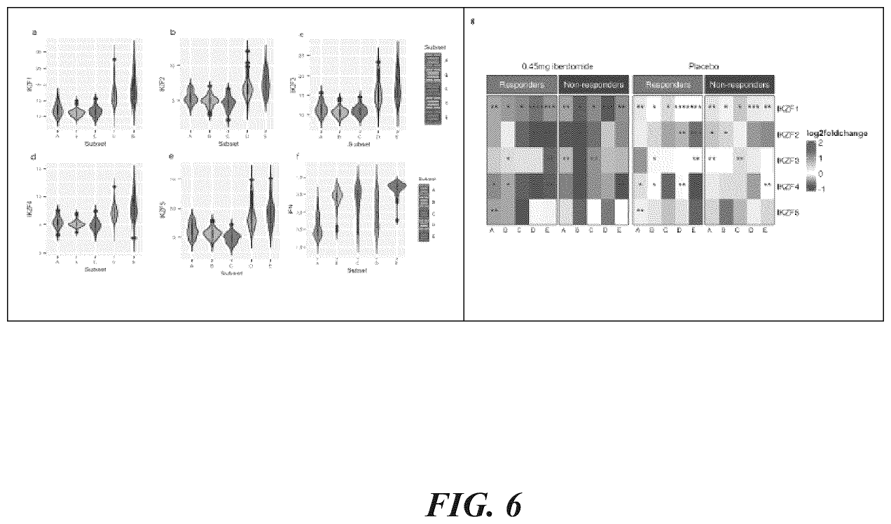

IKZF family member expression in lupus endotypes and change with iberdomide treatment. A previous analysis suggested that clinical responsiveness to iberdomide might relate to expression of Aiolos (10). We found that the pattern of expression of IKZF1-5 was similar in all 5 endotypes, with significantly higher expression of all in endotypes D and E (FIG. 6, left panel). Notably, expression of IKZF family members did not parallel the IGS (FIG. 6, right panel). Although the change in IKZF family members was somewhat variably affected by iberdomide therapy, in general, IKZF1-4, but not IKZF5 were most consistently decreased in endotype E responders (FIG. 6, left panel).

The heterogeneity of SLE pathogenesis represents a significant limitation in determining the optimal treatment for each individual patient. Moreover, heterogeneity confounds many clinical trials, making it difficult to determine whether tested therapies might be effective, but only in subsets of subjects. Although clinical trials demonstrated the overall safety and efficacy of the Ikaros and Aiolos-targeting drug iberdomide for the treatment of SLE subjects, the finding that not all trial participants exhibited a significant response points to a need for better understanding of the types of subjects that are more likely to benefit from iberdomide treatment. Gene expression-based molecular endotyping of lupus subjects to identify clinically relevant subsets with distinct immunologic features that were indicative of responsiveness to medication has been carried out. This study used the same immune pathway and cellular gene modules to categorize baseline blood samples from subjects in the iberdomide clinical trial into discrete endotypes and monitored the responsiveness of each endotype to treatment over time. Using this approach, subjects defined by molecular features that preferentially responded to iberdomide could be identified. Moreover, by tracking the transcriptomic features of the subjects longitudinally, it was noted that clinical responders exhibited changes in expression of specific gene clusters, whereas clinical nonresponders manifested minimal changes in gene expression profiles. These results may suggest that gene expression profiles at baseline predict clinical responsiveness, but also that the pharmacodynamic impact of iberdomide may vary in different subjects even within responsive subsets and appears to be necessary for a clinical response.

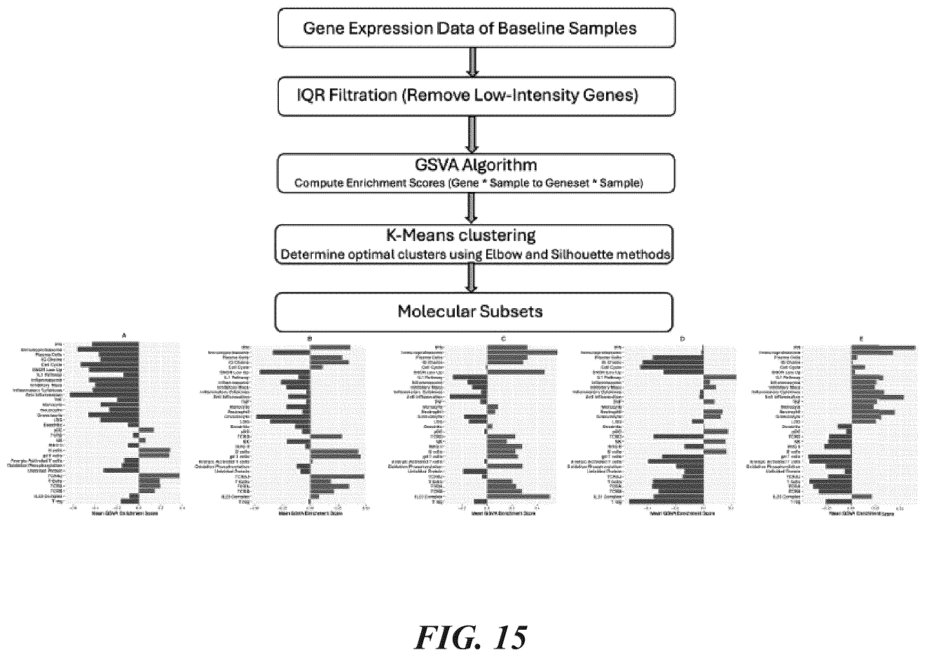

As an unbiased approach to identify patient endotypes, stable K-means clustering on GSVA scores of informative gene modules and delineated five lupus endotypes (A-E) were carried out, each characterized by a distinct molecular signature with endotype A as the least severe with the lowest number of molecular abnormalities and endotype E as the most severe with the highest number of molecular abnormalities indicative of heightened immune activity (FIG. 15). The demographic distribution within each endotype also provided insight into molecular features associated with patient ancestry. Endotypes B and C had a greater prevalence of individuals of African ancestry and, as we previously found, were characterized by more enrichment of plasma cells and Ig chains (17). Endotype E had the highest frequency of Native American subjects who are affected by SLE at a higher frequency and with greater clinical severity than subjects of European ancestry (19). These demographic associations may reflect genetic or environmental factors influencing molecular profiles and disease manifestation in SLE.

These endotypes derived from K-means clustering were also compared to our previous analysis of over 3,000 lupus subjects divided into eight endotypes through an ML-based approach. As a result, it was found that there was a high degree of similarity in the least severe endotype (A) and most severe endotypes (E and H) between the two approaches and this was also reflected in the treatment response observed in the most severe endotypes derived from both approaches. Notably, there was greater heterogeneity in the intermediate endotypes in that K means clustering divided these subjects between three endotypes and ML between five endotypes emphasizing the need for expanded datasets to better define subjects with intermediate disease pathology.

A composite lupus severity composite score (LSCS) was generated using GSVA scores from all gene modules combined to classify immunologic activity and relate molecular and clinical features of disease (8). The development of a composite score provides a clinical metric that estimates a patient's overall level of immune-mediated inflammation. LSCS values, that integrate scores of multiple gene modules, were significantly positively correlated with markers of disease activity, such as anti-dsDNA levels and SLEDAI-2K scores, and negatively correlated with complement levels, further validating the relevance of these molecular profiles in understanding disease severity. LSCS values could be utilized as a new clinical metric, that reflects immunologic activity and correlates with disease activity as measured by SLEDAI-2K scores, but can also be broken down into immunological components represented by the individual gene modules, that contribute to disease.

Molecular endotyping not only deconvolutes the heterogeneity inherent in SLE but also holds substantial promise for predicting therapeutic responses in SLE subjects (23-24). Specifically, endotypes C and E showed significant clinical improvement with iberdomide, especially at the highest dose, indicating that these molecular profiles are more likely to benefit from this treatment. Notably, a significant clinical response was observed only in endotype E across all dose levels, highlighting a robust connection between specific molecular gene signatures and clinical outcomes. SHAP analysis identified the specific gene modules that were the greatest contributors to clinical response to iberdomide in endotypes E and C. In endotype E, responders showed strong contributions from Treg and B cell signatures. Responders from endotype E were also the only participants to exhibit a significant change in the IFN response gene signature as compared to non-responders. In endotype C, key features linked to response included oxidative phosphorylation and anergic T cell signatures. These findings provide insights into the molecular features that drive treatment efficacy and underscore the potential of using such signatures to predict patient response.

Longitudinal analysis revealed that iberdomide treatment led to common pharmacodynamic effects across all endotypes, such as increased Treg cell and decreased B cell signatures. Notably, similar treatment effects were observed on initial analysis of the phase 2 iberdomide clinical trial (9-11). However, even within the same endotype, clinical responders exhibited a more pronounced pharmacodynamic effect than nonresponders, which revealed an unexpected variation in pharmacodynamic effects even among subjects with similar molecular profiles. This result emphasizes the need to monitor subjects to ensure that they are obtaining a pharmacodynamic effect from treatment as this seems to be associated with and potentially essential for clinical response.

This molecular endotyping approach revealed distinct molecular profiles of individuals in endotypes C and E, who achieved a significant clinical response. Unique changes were observed in endotype E, including reductions in the IGS and various inflammatory pathways. Endotype C responders exhibited fewer gene expression changes, despite a significant clinical response, suggesting that clinical benefits may arise from overall modulation of disease rather than specific molecular alterations. These findings may indicate that while iberdomide induces consistent, pharmacodynamic effects on immune cell signatures, the molecular impact can vary depending on the initial endotype, potentially influencing treatment outcomes. Knowledge of endotype membership has the potential to improve selection of appropriate subjects for clinical trial recruitment and to better inform clinicians in the administration of therapies and improve patient care (25-26).

Ikaros (IKZF1) and Aiolos (IKZF3) are members of a family of transcription factors including Helios (IKZF2), Eos, and Pegasus (IKZF4-5) that altogether participate in a network of interactions with other family members and transcriptional regulators to influence gene expression (27). Most of the mechanistic work on this family has focused on downstream effects of Ikaros activity and not on transcriptional regulation of the Ikaros family at the gene level, particularly when it applies to the ability of other family members to compensate for the loss of another (28). In addition, previous reports found that the clinical impact of iberdomide treatment was greater in subjects with higher expression of IKZF3 and type-I IFN at baseline (10-11). In the present study, baseline gene expression of IKZF1-5 and enrichment of the IFN gene module was significantly higher in endotype E, with the highest degree of inflammatory module enrichment, and lower in endotype A, the least inflammatory endotype. Clinical response to iberdomide treatment was not reflected in differences in baseline expression of IKZF1-5 as compared to placebo, suggesting that the overall molecular profile of each subject may be more predictive of clinical response than Ikaros family expression alone. The changes in gene module enrichment that accompanied iberdomide treatment reflected outcomes of Ikaros and Aiolos mutation or inhibition including immunodeficiency (29), IFN production (30), and IL2 regulation (31).

There may be B cell and IFN-specific effects of belimumab and anifrolumab have, respectively as well as the decrease in inflammatory pathways and genes associated with treatment response. These effects may be associated with molecular profiles of treatment responders as a whole and may not account for the high heterogeneity among individual study participants. Utilizing molecular endotyping may present a more detailed examination of groups of patients most likely to respond favorably to treatment based on the gene expression profiles in their respective endotypes.

Thus, we employed K-means clustering to subset subjects resulting in 5 endotypes was used here. In subjects with endotypes other than C, the possible molecular contributors to the predicted treatment response were very clear and showed a discernible pattern with significant associations with gene expression changes. Studies in larger cohorts may be useful to continue studying these findings.

The results of this study demonstrate that molecular-based, unsupervised clustering approach using RNA gene expression data to stratify lupus subjects into distinct molecular endotypes based on unique genomic biomarkers may yield essential information regarding the abnormalities contributing to inflammation and immune system dysfunction in individual subjects. By applying the K-means algorithm to baseline lupus blood samples from the phase 2b iberdomide trial, we were able to identify a balanced number of subjects for classification into each of five endotypes. Clinical responsiveness to iberdomide was confined to two molecular subsets offering the possibility of enriching trials for responsive patients and selecting patients for specific therapies in clinical care. Longitudinal studies demonstrated that clinical responsiveness to iberdomide was observed in those in whom a pharmacodynamic effect of the drug was detected, affording the possibility of monitoring the molecular impact of a drug to guide effective therapy. Our findings could inform clinicians of the usefulness of molecular biomarkers to anticipate clinical response in the management of lupus. Furthermore, this approach may empower physicians with useful clinical information to predict the efficacy of a given therapeutic agent and make informed treatment choices. This patient stratification algorithm, when used in conjunction with the robust LSCS scoring system, offers a personalized medicine strategy that could be transformative in the management of lupus case.

FIG. 1. Five molecular endotypes characterized by distinct immunologic features in 276 female subjects with systemic lupus erythematosus (SLE) from the phase 2b randomized controlled trial of iberdomide. Endotypes were identified by K-means clustering of gene set variation analysis (GSVA) scores derived from baseline (pretreatment) gene expression profiles. Top, Heatmaps show clustering of baseline samples into five endotypes, ordered by the “least abnormal” to the “most abnormal” immunologic activity (endotypes A-E), based on whole blood molecular expression of 32 cellular and process gene modules. Bottom, Endotypes were assessed according to disease activity scores on the SLE Disease Activity Index 2000 (SLEDAI-2K), with dots representing individual subjects. Heatmaps show gene expression profiles in each endotype by treatment arm (placebo or one of three doses of iberdomide once daily over 24 weeks), prednisone (pred) and hydroxychloroquine (HCQ) usage, ancestry, and female gender. AA=African American/African ancestry; ASIAN=Asian ancestry; EA=White European ancestry; NatAme=Native American or Alaska Native ancestry; O=other ancestry; IFN=type I interferon; IL-1=interleukin-1; Macs=macrophages; TNF=tumor necrosis factor; LDG=low-density granulocyte; pDC=plasmacytoid dendritic cells; TCRD=T cell receptor delta chain; NK=natural killer cells; MHC II=major histocompatibility complex class II; gd T cells=γδ T cells; TCRAJ=T cell receptor AJ sequence; TCRA=T cell receptor alpha chain; TCRB=T cell receptor beta chain; T reg=regulatory T cells.

FIG. 2. Clinical response rates at 24 weeks according to composite scores on the Systemic Lupus Erythematosus (SLE) Responder Index 4 (SRI-4) in SLE subjects in each treatment group, by molecular endotype. *=P=0.002 by Fisher's exact test, for highest dose (0.45 mg) iberdomide group versus placebo group.

FIG. 3. Impact of the highest dose of iberdomide (0.45 mg once daily) on gene expression profiles in SLE subjects at weeks 4, 12 and 24, by molecular endotype. a) compared to placebo b) among clinically determined responders and non-responders. Heatmaps indicate differences in GSVA scores between groups, assessed using Hedges' g. Asterisks indicate significant gene expression changes at each time point in each treatment group, as determined by Welch's analysis of variance with pairwise comparisons. *=P<0.05; **=P<0.01; ***=P<0.001; ****=P<0.0001. See FIG. 1 for definitions.

FIG. 4. Impact of Iberdomide Treatment on Gene Expression Profiles of subset E Assessed by Lupus Severity Scores (LSCS). a) Gene-based scores at baseline, weeks 4, 12, and 24 are visualized for responders and non-responders in the placebo and high dose (0.45 mg iberdomide) groups using beeswarm plots. b) Statistical analysis of LSCS at baseline and week 24 gene expression profiles is presented for responders and non-responders of each treatment group. c) Correlation analysis of LSCS with SLEDAI in responders and non-responders with placebo and high-dose (0.45 mg) iderdomide is depicted, with the color of each dot representing the time point. d) A heatmap visualizes the correlation coefficients of GSVA scores from 32 gene modules with SLEDAI in responders and non-responders among placebo and iberdomide treated subjects from endotypes E and C. Red indicates the positive correlation coefficients, while blue indicates negative correlation coefficients with SLEDAI. *=P<0.05; **=P<0.01; **=P<0.001; ****=P<0.0001.

FIG. 5. Heatmaps showing the key contributing features to the random forest (RF) model predictions of treatment response to the highest dose of iberdomide (0.45 mg once daily), identified using Shapley Additive explanations (SHAP) analysis. SHAP values of the top unique features from the RF classifier were constructed between baseline and week 24 lupus blood samples from responder subjects with endotype E (top panels) or endotype C (bottom panels) who received the highest dose of iberdomide. The f(x) line at top represents the model prediction outcome value for each feature in each subject. The higher the f(x) value, the closer the model predicts the sample as positive class (at week 24). Heatmaps were generated using the SHAP module in Python. See FIG. 1 for other definitions.

FIG. 6. Transcriptomics analysis showing changes in gene expression of Ikaros zinc finger (IKZF) transcription factors and type I interferon (IFN) by endotype in blood samples from systemic lupus erythematosus (SLE) subjects after 24 weeks of treatment with the highest dose of iberdomide (0.45 mg once daily) or placebo. Left, The log₂ fold change in expression of IKZF1 (a), IKZF2 (b), IKZF3 (c), IKZF4 (d), and IKZF5 (e), as well as expression of IFN genes (f), after treatment with high-dose iberdomide is summarized as violin plots for each of the five endotypes derived from K-means clustering. The bar graphs were generated in R using ggplot2. Right, Heatmaps show the changes in gene expression in the responder and nonresponder groups of subjects by endotype in the high-dose iberdomide and placebo groups (g). Significant changes were determined by t-test. *=P<0.05; **=P<0.01; ***=P<0.001; ****=P<0.0001).

FIG. 7. Elbow plots (a) and silhouette plots (b) derived using the K-means algorithm to determine the optimal number of clusters from baseline lupus blood samples, a, by fitting the model on the range of K values, the data based on distortion scores in the elbow plot suggest that the optimal number (K) of clusters is five. The elbow plot was generated using KElbowvisualizer in scikit-learn in Python. b, Silhouette plots show the five cluster labels (clusters 0-4), annotated as follows: cluster 0=A; cluster 1=E; cluster 2=D; cluster 3=B; cluster 4=C. The coefficient values for each cluster represent the quality of clustering. The silhouette plot of K-means clustering data in five centers was plotted using matplotlib in scikit-learn in Python. c, PCA plot of clinical features (age, SLEDAI, BILAG, C3, C4, anti-dsDNA, CLASIA, and CLASID) and categorical features (ancestry, RNP, SSA, SSB, and anti-SM) from baseline study data. Data points are colored based on SRI-4 clinical response.

FIG. 8. Baseline clinical characteristics of the subjects in the five lupus molecular endotypes derived from K-means clustering. a-d, Violin plots show that anti-double-stranded DNA (anti-dsDNA) levels (a) and Systemic Lupus Erythematosus Disease Activity Index 2000 (SLEDAI-2K) scores (d) were positively correlated with the Lupus Severity Composite Score (LSCS), while serum levels of C3 and C4 were inversely proportional to the LSCS (b and c). e-h, The relationship between LSCS scores and each clinical feature was assessed using Pearson's correlation analyses. Each dot represents an individual lupus blood sample, and the color of the dot corresponds to the indicated molecular subset.

FIG. 9 Machine learning analysis of gene set variation analysis (GSVA) scores from 276 baseline lupus blood samples using random forest (RF) model weights. The RF model was constructed on 3,166 baseline lupus blood samples using 26 GSVA scores of gene modules. Top. Heatmaps show clustering of the baseline samples into eight endotypes (A-H) based on whole blood molecular expression of 32 cellular and process gene modules. Heatmaps were generated using ComplexHeatmap (Bioconductor package in R). Bottom. Data were assessed according to disease activity scores on the SLE Disease Activity Index 2000 (SLEDAI-2K), with dots representing individual subjects. Heatmaps show gene expression profiles in each endotype by treatment arm, prednisone (pred) usage, hydroxychloroquine (HCQ) usage, race/ancestry, and female gender. AA=African American/African ancestry; ASIAN=Asian ancestry; EA=White European ancestry; NatAme=Native American or Alaska Native ancestry; O=other ancestry; IFN=type I interferon; IL-1=interleukin-1; Macs=macrophages; TNF=tumor necrosis factor; LDG=low-density granulocyte; pDC=plasmacytoid dendritic cells; TCRD=T cell receptor delta chain; NK=natural killer cells; MHC II=major histocompatibility complex class II; gd T cells=γδ T cells; TCRAJ=T cell receptor AJ sequence; TCRA=T cell receptor alpha chain; TCRB=T cell receptor beta chain; T reg=regulatory T cells.

FIG. 11. Comparison of the random forest (RF) classifier model and K-means clustering approach to identify lupus molecular endotypes. A confusion matrix was constructed to elucidate the congruence in patient classification between the two methods. Data are the Adjusted Rand Index of concordance, stratified by endotype.

FIG. 12. Clinical responses of subjects in the random forest-derived subsets (endotypes) defined by gene expression profiles. Clinical responses to placebo or one of three doses of iberdomide were derived from composite scores on the Systemic Lupus Erythematosus Responder Index 4 (SRI-4) in subjects categorized into eight endotypes based on gene expression profiles. Notably, the highest dose (0.45 mg once daily) iberdomide group displayed significant response rates as compared to the placebo group in

FIG. 13. Impact of high dose iberdomide treatment on gene expression profiles in subjects with endotypes A (a), C (b) and E (c) among responders in each of the four treatment groups at weeks 4, 12 and 24. Asterisks indicate the significant gene expression changes at each time point in each treatment group, as determined by paired t-test. *=P<0.05; **=P<0.01; =P<0.001; ****=P<0.0001.

FIG. 14. Heatmaps showing the key contributing features to the random forest (RF) model predictions of treatment response to placebo, identified using Shapley Additive explanations (SHAP) analysis. SHAP values of the top unique features from the RF classifier were constructed between baseline and week 24 lupus blood samples from responder subjects with endotype E (a) or endotype C (b) who received placebo. The f(x) line at top represents the model prediction outcome value for each feature in each subject. The higher the f(x) value, the closer the model predicts the sample as positive class (at week 24). Heatmaps were generated using the SHAP module in Python.

FIG. 15. Flow diagram of the pipeline for generation of molecular 5 molecular endotypes from the phase 2b iberdomide clinical trial. Gene module enrichments for each endotype are summarized in bar plots where red bars indicate modules with increased enrichment and blue bars indicate modules with decreased enrichment.

1-45. (canceled)

46. A method of monitoring treatment of a subject, the method comprising:
(a) providing a first sample from the subject having received a first dose of a first cereblon E3 ligase modulator;
(b) assaying the first sample to determine expression levels of genes of a plurality of gene sets in the first sample, wherein the plurality of gene sets comprises at least one gene set comprising at least 30% of genes of a gene set from Table 1;
(c) determining a first endotype of the subject, based at least in part on the expression levels determined in (b); and
(d) based at least in part on the first endotype, determining a second dose of a second cereblon E3 ligase modulator for administration to the subject.

47. The method of claim 46, further comprising, prior to (a), providing a second sample from the subject.

48. The method of claim 47, further comprising assaying the second sample to determine second expression levels of genes of the plurality of gene sets, and determining a second endotype of the subject, based at least in part on the second expression levels.

49. The method of claim 48, further comprising determining the first dose of the first cereblon E3 ligase modulator, based at least in part on the second endotype.

50. The method of claim 49, further comprising administering the first dose of the first cereblon E3 ligase modulator to the subject.

51. The method of claim 46, further comprising, subsequent to (a), processing the first sample to produce an enriched sample.

52. The method of claim 51, wherein the processing comprises enriching the first sample for at least a subset of the genes in the plurality of gene sets.

53. The method of claim 52, wherein the enriching comprises using a probe set to bind to the at least the subset of the genes in the plurality of gene sets.

54. The method of claim 46, further comprising, subsequent to (c), administering the second dose of the second cereblon E3 ligase modulator to the subject.

55. The method of claim 46, further comprising, subsequent to (c), generating an electronic report for a care provider indicative of the second dose of the second cereblon E3 ligase modulator.

56. The method of claim 46, wherein the subject has or is suspected of having an autoimmune disease.

57. The method of claim 56, wherein the autoimmune disease is lupus.

58. The method of claim 46, wherein (b) further comprises performing ribonucleic acid sequencing (RNA-seq).

59. The method of claim 46, wherein the plurality of gene sets comprises at least 6 gene sets.

60. The method of claim 59, wherein the first sample comprises cell lysate from white blood cells, cell-free RNA, or a combination thereof.

61. The method of claim 46, wherein the first cereblon E3 ligase modulator comprises thalidomide, lenalidomide, pomalidomide, mezigdomide, iberdomide, golcadomide, or an analog thereof.

62. The method of claim 46, wherein the first cereblon E3 ligase modulator comprises iberdomide.

63. The method of claim 46, wherein the first dose of the first cereblon E3 ligase modulator comprises an oral dose.

64. The method of claim 46, wherein the second cereblon E3 ligase modulator comprises iberdomide.

65. The method of claim 46, wherein the second dose of the second cereblon E3 ligase modulator comprises an oral dose.
